(19) United States
(12) Patent Application Publication
(10) Pub. No.: US 2012/0171118 A1
(43) Pub. Date: Jul. 5, 2012

(54) TREATMENT OF MENINGEAL AND NEURAL DISEASES

(75) Inventor: Mikhail I. Papisov, Winchester, MA (US)

(73) Assignee: THE GENERAL HOSPITAL CORPORATION, Boston, MA (US)

(21) Appl. No.: 13/376,915

(22) PCT Filed: Jun. 11, 2010
(86) PCT No.: PCT/US10/38430

§ 371 Date: Mar. 23, 2012

Related U.S. Application Data
(60) Provisional application No. 61/186,806, filed on Jun. 12, 2009.

(51) Int. Cl. A61K 31/4745; A61P 25/00; A61P 35/00; A61P 31/00; A61P 25/28; A61P 7/06; A61P 25/16; A61P 27/02; A61K 51/04; A61P 35/04

(52) U.S. Cl. 424/1.85; 514/283

(57) ABSTRACT

The present invention provides conjugates and methods of using the same for the treatment of cerebral, meningeal, and neural diseases, disorders, and conditions. Provided methods include administering conjugates directly into the cerebrospinal fluid space of an animal.

### CROSS REFERENCE TO RELATED APPLICATIONS

The present invention claims priority to U.S. provisional patent application Ser. No. 61/186,806, filed Jun. 12, 2009, the entirety of which is hereby incorporated by reference.

### FIELD OF THE INVENTION

This invention relates to the field of diagnosis and treatment of the diseases of brain, spinal cord, large nerves, meninges, and other tissues directly or indirectly contacting cerebrospinal fluid.

### BACKGROUND OF THE INVENTION

The brain, spinal cord, large nerves and the surrounding meninges are notoriously inaccessible for systemically administered pharmaceutical agents due to both the blood-brain barrier (BBB) and the blood-cerebrospinal fluid (CSF) barrier. Direct administration of conventional drugs into CSF is well studied but for the most part inefficient outside anesthesiology, presumably due to the fast clearance of the drug from CSF into systemic circulation. Meningeal cancer is particularly resistant to the available treatments. There are several other conditions, including life-threatening ones, which could be more effectively treated if drugs capable of long residency in CSF were available.

### BRIEF DESCRIPTION OF THE DRAWINGS

FIG. 1 depicts the distribution of RNAse in the CSF four minutes after intrathecal administration into the cisterna magne (cross: administration site). Transverse, coronal and saggital 1 mm slices through the injection point. Double-headed arrows: brain area (arachnoidal space and ventricles); Single-headed solid arrow: spinal cord; Single-headed dashed arrow: olfactory nerves.

FIG. 2 depicts the translocation of RNAse in the CSF over twenty minutes post-injection. Line 1: radioactivity in the cisterna magna; Line 2: radioactivity in the right ventricle; Line 3: radioactivity in the area of the distal segment of the olfactory nerve; Line 4: radioactivity in the area of the proximal segment of the olfactory nerve; Line 5: radioactivity in the spinal cord area.

FIG. 3 depicts the distribution of BSA four hours post-injection into the cisterna magna. Transverse, coronal and saggital 1 mm slices through the point marked by cross. Double-headed arrows: brain area (arachnoidal space and ventricles); Single-headed solid arrow: the olfactory nerve pair region; Single-headed dashed arrow: olfactory bulbs area.

FIG. 4 depicts the distribution of RNAse four hours post-injection into the cisterna magna. Transverse, coronal and saggital 1 mm slices through mid-brain (marked by cross). Dashed arrow: brain area (arachnoidal space and ventricles); Solid arrow: ventricles; Wide, solid arrow: olfactory bulbs area; Dashed arrow: thyroid (accumulation of ¹²⁴I released as a result of protein digestion).

FIG. 5 depicts the anatomy of neoplastic meningitis.

FIG. 6 depicts typical retention profiles of molecular filters with varying pore size distribution. C: the cutoff range.

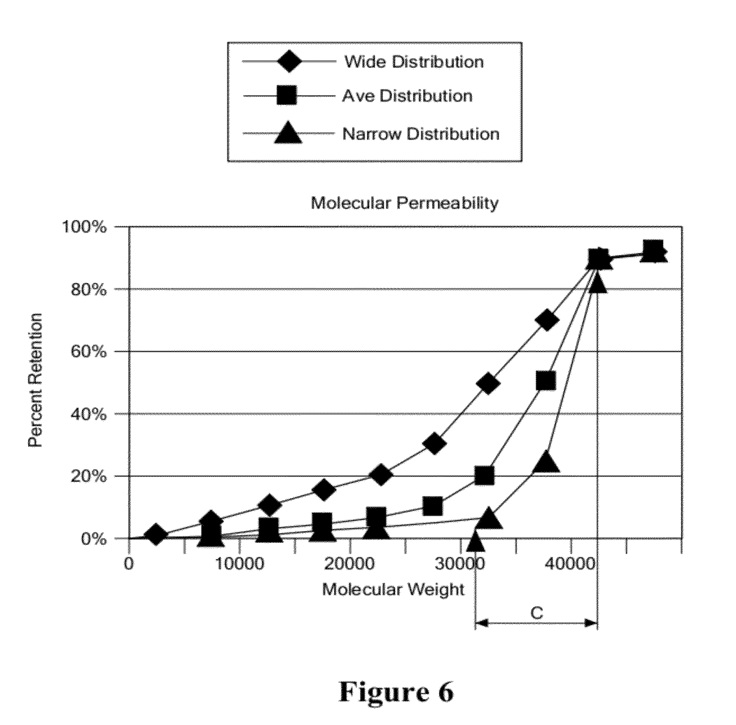

FIG. 7 depicts distribution of CPT-PHF conjugate in the cranial CSF volume and spinal CSF immediately after the injection. PET imaging, 0-20 minutes after intrathecal administration into cysterna magna. Inverted black and white linear scale. Top to bottom: animals 1, 2 and 3. Columns: Left: transverse slice, Central: coronal slice, Right: sagittal slice, Slice thickness: 0.64 mm. White cursor cross depicts slice position in all dimensions.

FIG. 8 depicts residual content of CPT in the cranial CSF volume and spinal CSF 2 hours after the injection of the CPT-PHF conjugate. PET imaging, 2 hr to 2 hr, 20 minutes after intrathecal administration into cysterna magna. Inverted black and white linear scale. Top to bottom: animals 1, 2 and 3. Columns: Left: transverse slice, Central: coronal slice, Right: sagittal slice, Slice thickness: 0.64 mm. White cursor cross depicts slice position in all dimensions.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference.

The term “biocompatible”, as used herein is intended to describe compounds that exert minimal destructive or host response effects while in contact with body fluids or living cells or tissues. Thus a “biocompatible group”, as used herein, refers to an aliphatic, heteroaliphatic, aryl or heteroaryl moiety, which falls within the definition of the term “biocompatible,” as defined above and herein. The term “Biocompatibility” as used herein, is also taken to mean minimal interactions with recognition proteins, e.g., naturally occurring antibodies, cell proteins, cells and other components of biological systems, unless such interactions are specifically desirable. Thus, substances and functional groups specifically intended to cause the above effects, e.g., drugs and prodrugs, are considered to be biocompatible. In certain embodiments (with exception of compounds intended to be cytotoxic, such as e.g. antineoplastic agents), compounds are “biocompatible” if their addition to normal cells in vitro, at concentrations similar to the intended systemic in vivo concentrations, results in less than or equal to 1% cell death during the time equivalent to the half-life of the compound in vivo (e.g., the period of time required for 50% of the compound administered in vivo to be eliminated/cleared), and their administration in vivo induces minimal and medically acceptable inflammation, foreign body reaction, immunotoxicity, chemical toxicity, or other such adverse effects. In the above sentence, the term “normal cells” refers to cells that are not intended to be destroyed or otherwise significantly affected by the compound being tested.

As used herein, “biodegradable” compound are compound that are susceptible to biological processing in vivo. As used herein, “biodegradable” compounds are those that, when taken up by cells, can be broken down by the lysosomal or other chemical machinery or by hydrolysis into components that cells can either reuse or dispose of without significant toxic effect on the cells. Degradation fragments preferably induce no or little organ or cell overload or pathological processes caused by such overload or other adverse effects in vivo. Examples of biodegradation processes include enzymatic and non-enzymatic hydrolysis, oxidation and reduction. Suitable conditions for non-enzymatic hydrolysis of polymer backbones of various conjugates, for example, include exposure of biodegradable conjugates to water at a temperature and a pH of lysosomal intracellular compartment. Biodegradation of some conjugate backbones can also be enhanced extracellularly (e.g. in low pH regions of the animal body such as an inflamed area), in the close vicinity of activated macrophages or other cells releasing degradation facilitating factors. In certain embodiments, the effective size of a polymer molecule at pH ˜7.5 does not detectably change over 1 to 7 days, and remains within 50% of the original polymer size for at least several weeks. In certain embodiments, at pH ˜5 the polymer detectably degrades over 1 to 5 days and is completely transformed into low molecular weight fragments within a two-week to several-month time frame. Polymer integrity in such tests can be measured, for example, by size exclusion HPLC. In some embodiments, faster degradation is desired. In certain embodiments a polymer degrades in cells with the rate that does not exceed the rate of metabolization or excretion of polymer fragments by the cells. In some embodiments, polymers and polymer biodegradation byproducts are biocompatible.

The term “hydrophilic” as it relates to substituents on a carrier (e.g., polymer monomeric units) does not essentially differ from the common meaning of this term in the art, and denotes chemical entities or moieties which contain ionizable, polar, or polarizable atoms, or which otherwise may be solvated by water molecules. Thus a “hydrophilic group,” as used herein, refers to an aliphatic, heteroaliphatic, aryl or heteroaryl moiety, which falls within the definition of the term “hydrophilic,” as defined above. Examples of particular hydrophilic organic moieties include, without limitation, aliphatic or heteroaliphatic groups comprising a chain of atoms in a range of between about one and twelve atoms, hydroxyl, hydroxyalkyl, amine, carboxyl, amide, carboxylic ester, thioester, aldehyde, nitryl, isonitryl, nitroso, hydroxylamine, mercaptoalkyl, heterocycle, carbamates, carboxylic acids and their salts, sulfonic acids and their salts, sulfonic acid esters, phosphoric acids and their salts, phosphate esters, polyglycol ethers, polyamines, polycarboxylates, polyesters and polythioesters. In some embodiments, a hydrophilic group of a polymer monomeric unit is a carboxyl group (COOH), an aldehyde group (CHO), a methylol (CH₂OH) group, ethylol (CH₂CH₂OH) group, propylol (CH₂CH₂CH₂OH) group or a glycol (for example, CHOH—CH₂OH or CH—(CH₂OH)₂) group.

The term “hydrophilic” as it relates to the carriers of the invention generally does not differ from usage of this term in the art, and denotes carriers comprising hydrophilic functional groups as defined above. In some embodiments, such carriers are polymers. In some embodiments, a hydrophilic polymer is a water-soluble polymer. In certain embodiments, a hydrophilic polymer is a polyacetal or polyketal. Hydrophilicity of the carrier can be directly measured through determination of hydration energy, or determined through investigation between two liquid phases, or by chromatography on solid phases with known hydrophobicity, such as, for example, C4 or C18.

The term “biomolecules”, as used herein, refers to molecules (e.g., proteins, amino acids, peptides, polynucleotides, nucleotides, carbohydrates, sugars, lipids, nucleoproteins, glycoproteins, lipoproteins, steroids, etc.) which belong to classes of chemical compounds, whether naturally-occurring or artificially created (e.g., by synthetic or recombinant methods), that are commonly found in cells and tissues. Examplary types of biomolecules include, but are not limited to, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, DNA, and RNA.

The term “carrier”, as used herein, refers to any large molecule, macromolecule, biomolecule, particle, gel or other object or material which is or can be covalently attached to one or more modifier molecules with a suitable linker. In certain embodiments, a carrier prolongs a time of residence in the CSF for a modifier molecule with which it is associated. In certain embodiments, a carrier is a polymer (e.g., a synthetic polymer, a naturally-occurring polymer, a chemically modified naturally occurring polymer, etc.).

The phrase “physiological conditions”, as used herein, relates to the range of chemical (e.g., pH, ionic strength) and biochemical (e.g., enzyme concentrations) conditions likely to be encountered in the extracellular fluids of living tissues. For most normal tissues, the physiological pH ranges from about 7.0 to 7.4. Circulating blood plasma, cerebrospinal fluid, and normal interstitial liquid represent typical examples of normal physiological conditions.

The term “polyal” means a polymer having at least one acetal or ketal oxygen atom in each monomer unit positioned within the main chain. Examples of polyals can be found in U.S. Pat. Nos. 5,811,510, 5,863,990, 5,958,398; U.S. Patent Application Publication Nos. 2006/0069230 and 2007/0190018; and International Application Publication No. WO/2005/023294, each of which are incorporated herein by reference in their entirety. In certain embodiments, biodegradable biocompatible polymer carriers, useful for preparation of polymer conjugates described herein, are naturally occurring polysaccharides, glycopolysaccharides, and synthetic polymers of polyglycoside, polyacetal, polyamide, polyether, and polyester origin and products of their oxidation, functionalization, modification, cross-linking, and conjugation. In certain embodiments, a polyal is a polyacetal or polyketal that is substantially free of the cyclic acetal or ketal units that are contained in the parent polysaccharide or other polymer from which the polyal is derived. When the monomer units of a polyal are depicted herein the two free hydroxyls therein are equally reactive during derivitization and therefore either hydroxyl may be actually derivatized not just the one depicted.

The terms “polysaccharide”, “carbohydrate”, or “oligosaccharide” are known in the art and refer, generally, to substances having chemical formula (CH₂O)ₙ, where n>2, and their derivatives. Carbohydrates are polyhydroxyaldehydes or polyhydroxyketones, or change to such substances on simple chemical transformations, such as hydrolysis, oxydation or reduction. Typically, carbohydrates are present in the form of cyclic acetals or ketals (such as, glucose or fructose). These cyclic units (monosaccharides) may be connected to each other to form molecules with few (oligosaccharides) or several (polysaccharides) monosaccharide units. Often, carbohydrates with well defined number, types and positioning of monosaccharide units are called oligosaccharides, whereas carbohydrates consisting of mixtures of molecules of variable numbers and/or positioning of monosaccharide units are called polysaccharides. The terms “polysaccharide”, “carbohydrate”, and “oligosaccharide”, are used herein interchangeably. A polysaccharide may include natural sugars (e.g., glucose, fructose, galactose, mannose, arabinose, ribose, and xylose) and/or derivatives of naturally occurring sugars (e.g., 2′-fluororibose, 2′-deoxyribose, and hexose).

As used herein, the term “small molecule” refers to molecules, whether naturally-occurring or artificially created (e.g., via chemical synthesis) that have a relatively low molecular weight. In some embodiments, small molecules are biologically active in that they produce a local or systemic effect in animals, preferably mammals, more preferably humans. In some embodiments, small molecules have a molecular weight of less than about 1500 Da (1500 g/mol). In some embodiments, small molecules may be characterized by the hydrodynamic diameter. Thus in some embodiments a small molecule has a hydrodynamic diameter of less than 1 nm. In some embodiments, a small molecule has a hydrodynamic diameter of less than 12 nm. In certain embodiments, a small molecule has a hydrodynamic diameter of up to 50 nm. In certain embodiments, the small molecule is a drug and the small molecule is referred to as “drug molecule” or “drug”. In certain embodiment, the drug molecule has MW smaller or equal to about 1 kDa. In certain embodiments, the drug is one that has already been deemed safe and effective for use by the appropriate governmental agency or body. For example, drugs for human use listed by the FDA under 21 C.F.R. §§330.5, 331 through 361, and 440 through 460; drugs for veterinary use listed by the FDA under 21 C.F.R. §§500 through 589, incorporated herein by reference, are all considered suitable for use with the present invention.

Classes of drug molecules that can be used in the practice of the present invention include, but are not limited to, central nervous system-active agents, anti-cancer substances, radionuclides, paramagnetic entities, vitamins, anti-AIDS substances, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, and imaging agents. Alternatively and/or additionally, drugs encompass “large molecules” or macromolecules that have therapeutic value.

A more complete, although not exhaustive, listing of classes and specific drugs suitable for use in the present invention may be found in “Pharmaceutical Substances: Syntheses, Patents, Applications” by Axel Kleemann and Jurgen Engel, Thieme Medical Publishing, 1999 and the “Merck Index: An Encyclopedia of Chemicals, Drugs, and Biologicals”, Edited by Susan Budavari et al., CRC Press, 1996, both of which are incorporated herein by reference.

As used herein, the term “pharmaceutically useful group or entity” refers to a compound or fragment thereof, or a chemical moiety which, when associated with conjugates of the present invention, can exert some biological or diagnostic function or activity when administered to a subject, enhance the therapeutic, diagnostic or preventive properties of the conjugates in biomedical applications, improve safety, alter biodegradation or excretion, or is detectable. Examples of suitable pharmaceutically useful groups or entities include hydrophilicity/hydrophobicity modifiers, pharmacokinetic modifiers, biologically active modifiers, and detectable modifiers.

As used herein, the term “modifier” refers to an organic, inorganic or bioorganic moiety that is associated with a carrier. Modifiers can be small molecules or macromolecules, and can belong to any chemical or pharmaceutical class, e.g., nucleotides, chemotherapeutic agents, antibacterial agents, antiviral agents, immunomodulators, hormones or analogs thereof, enzymes, inhibitors, alkaloids and therapeutic radionuclides a therapeutic radionuclide (e.g., alpha, beta or positron emitter). In certain embodiments, chemotherapeutic agents include, but are not limited to, topoisomerase I and II inhibitors, alkylating agents, anthracyclines, doxorubicin, cisplastin, carboplatin, vincristine, mitromycine, taxol, camptothecin, antisense oligonucleotides, ribozymes, and dactinomycines. In certain embodiments, modifiers according to the invention include, but are not limited to, biomolecules, small molecules, therapeutic agents, pharmaceutically useful groups or entities, macromolecules, diagnostic labels, chelating agents, hydrophilic moieties, dispersants, charge modifying agents, viscosity modifying agents, surfactants, coagulation agents and flocculants, to name a few. A modifier can have one or more pharmaceutical functions, e.g., biological activity and pharmacokinetics modification. Pharmacokinetics modifiers can include, for example, antibodies, antigens, receptor ligands, hydrophilic, hydrophobic, or charged groups. Biologically active modifiers include, for example, drugs and prodrugs, antigens, immunomodulators. Detectable modifiers include diagnostic labels, such as radioactive, fluorescent, paramagnetic, superparamagnetic, ferromagnetic, X-ray modulating, X-ray-opaque, ultrosound-reflective, and other substances detectable by one of available clinical or laboratory methods, e.g., scintigraphy, NMR spectroscopy, MRI, X-ray tomography, sonotomography, photoimaging, radioimmunoassay. Viral and non-viral gene vectors are considered to be modifiers.

As used herein, the term “macromolecule” refers to molecules, whether naturally-occurring or artificially created (e.g., via chemical synthesis) that have a relatively high molecular weight, generally above 1500 g/mole. In some embodiments, macromolecules are biologically active in that they exert a biological function in animals, preferably mammals, more preferably humans. Examples of macromolecules include proteins, lipids, polyelectrolytes, polypeptides, polynucleotides, and/or polysaccharides. For the purpose of this invention, supramolecular constructs such as viruses, nucleic acid helices and protein associates (e.g., dimers or higher order complexes) are considered to be macromolecules. When associated with the conjugates of the invention, a macromolecule may be chemically or non-covalently modified prior to being associated with said conjugate. In certain embodiments, where macromolecules are associated with a carrier that prolongs their residence in the CSF, biologically active macromolecules and supramolecular constructs may act as and/or be considered “modifiers”, as described herein.

As used herein, the term “diagnostic label” refers to an atom, group of atoms, moiety or functional group, a nanocrystal, or other discrete element of a composition of matter, that can be detected in vivo or ex vivo using analytical methods known in the art. When associated with a conjugate of the present invention, such diagnostic labels permit the monitoring of the conjugate in vivo. Alternatively or additionally, constructs and compositions that include diagnostic labels can be used to monitor biological functions or structures. Examples of diagnostic labels include, without limitation, labels that can be used in medical diagnostic procedures, such as, radioactive isotopes (radionuclides) for gamma scintigraphy and Positron Emission Tomography (PET), contrast agents for Magnetic Resonance Imaging (MRI) (for example paramagnetic atoms and superparamagnetic nanocrystals), contrast agents for computed tomography and other X-ray-based imaging methods, agents for ultrasound-based diagnostic methods (sonography), agents for neutron activation (e.g., boron, gadolinium), fluorophores for various optical procedures, and, in general moieties which can emit, reflect, absorb, scatter or otherwise affect electromagnetic fields or waves (e.g., gamma-rays, X-rays, radiowaves, microwaves, light), particles (e.g., alpha particles, electrons, positrons, neutrons, protons) or other forms of radiation, e.g., ultrasound.

“Protecting groups” are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference.

“Protected hydrophilic group” and “protected organic moiety” as used herein, mean a hydrophilic group or organic moiety modified with a protecting group which will not interfere with a chemical reaction that a carrier or carrier conjugate is subjected to. Examples of protected hydrophilic groups include those described by Greene (supra), such as carboxylic esters, alkoxy groups, thioesters, thioethers, haloalkyl groups, Fmoc-alcohols, etc.

The term “aliphatic” or “aliphatic group,” as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-20 carbon atoms. In some embodiments, aliphatic groups contain 1-10 carbon atoms. In some embodiments, aliphatic groups contain 1-8 carbon atoms. In some embodiments, aliphatic groups contain 1-6 carbon atoms, and in some embodiments aliphatic groups contain 1-4 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon. This includes any oxidized form of nitrogen, sulfur, phosphorus, or silicon; the quaternized form of any basic nitrogen, or; a substitutable nitrogen of a heterocyclic ring including ═N— as in 3,4-dihydro-2H-pyrrolyl, —NH— as in pyrrolidinyl, or ═N(R^{†})— as in N-substituted pyrrolidinyl.

The term “unsaturated,” as used herein, means that a moiety has one or more units of unsaturation.

The term “aryl” used alone or as part of a larger moiety as in “aralkyl,” “aralkoxy,” or “aryloxyalkyl,” refers to monocyclic, bicyclic, and tricyclic ring systems having a total of five to fourteen ring members, wherein at least one ring in the system is aromatic and wherein each ring in the system contains three to seven ring members. The term “aryl” may be used interchangeably with the term “aryl ring.”

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 3- to 12-membered monocyclic or 7-12-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable trivalent substituents include, without limitation, acetylene, nitrile and isonitrile bonds, which may be coordinated with metals. A suitable tetravalent substituent that is bound to vicinal substitutable methylene carbons of an “optionally substituted” group is the dicobalt hexacarbonyl cluster represented by

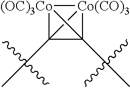

when depicted with the methylenes which bear it.

Suitable substituents on the aliphatic group of R* include halogen, —R^{•}, —(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “PHF” means [poly-(1-hydroxymethylethylene hydroxyl-methyl formal)].

The term “animal”, as used herein, refers to humans as well as non-human animals, at any stage of development, including, for example, mammals, birds, reptiles, amphibians, fish In some embodiments, a non-human animal is a mammal (e.g., a rodent, a mouse, a rat, a rabbit, a monkey, a dog, a cat, a primate, or a pig). An animal may be a transgenic animal or a human clone. The term “subject” encompasses animals.

When two entities are “associated with” one another as described herein, they are linked by a direct or indirect covalent or non-covalent interaction.

As it refers to an active agent or drug delivery device, the term “efficient amount” refers to the amount necessary to elicit the desired biological response. As will be appreciated by those of ordinary skill in this art, the efficient amount of an agent, modifier, or device may vary depending on such factors as a desired biological endpoint, an agent to be delivered, a composition of an encapsulating matrix, a target tissue, etc.

As used herein, the term “directly attached”, as it refers to covalent attachment of one entity to another, means that the two entities are connected via a covalent bond. In certain embodiments, the present disclosure describes modifiers attached to carriers via linkers, whereby the point of attachment comprises a cleavable bond.

As used herein, the term “indirectly attached”, as it refers to association of one entity to another, means that the two entities are connected via a linking moiety (as opposed to a direct covalent bond). Example of non-covalent interactions include, without limitation, hydrogen bonding, van der Waals interactions, hydrophobic interactions, magnetic interactions, electrostatic interactions, or combinations thereof, etc.

The term “natural amino acyl residue” as used herein refers to any one of the common, naturally occurring L-amino acids found in naturally occurring proteins: glycine (Gly), alanine (Ala), valine (Val), leucine (Leu), isoleucine (Ile), lysine (Lys), arginine (Arg), histidine (His), proline (Pro), serine (Ser), threonine (Thr), phenylalanine (Phe), tyrosine (Tyr), tryptophan (Trp), aspartic acid (Asp), glutamic acid (Glu), asparagine (Asn), glutamine (Gln), cysteine (Cys) and methionine (Met).

The term “unnatural amino acyl residue” as used herein refers to any amino acid which is not a natural amino acid. This includes, for example, α-, β-, ω-, D-, and L-amino acyl residues. Such residues include the D-isomer of any of the 20 naturally occurring amino acids. Unnatural amino acids include homoserine, ornithine, norleucine, and thyroxine. Other unnatural amino acid side-chains are well known in the art and include side chains that are N-alkylated, cyclized, phosphorylated, acetylated, amidated, azidylated, labelled, and the like.

As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition for the purpose of decreasing the risk of developing pathology associated with the disease, disorder, and/or condition.

The expression “unit dose” as used herein refers to a physically discrete unit of a formulation appropriate for a subject to be treated. It will be understood, however, that the total daily usage of a formulation of the present invention will be decided by the attending physician within the scope of sound medical judgment. The specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts. A particular unit dose may or may not contain a therapeutically effective amount of a therapeutic agent.

An individual who is “suffering from” a disease, disorder, and/or condition has been diagnosed with and/or displays one or more symptoms of the disease, disorder, and/or condition.

An individual who is “susceptible to” a disease, disorder, and/or condition has not been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

### DETAILED DESCRIPTION OF CERTAIN EMBODIMENTS

The present invention provides, among other things, safe and effective treatments for diseases and disorders affecting the meninges and central nervous system. In certain embodiments, a conjugate as described herein is administered directly into the cerebrospinal fluid space of a patient to treat one or more diseases or disorders affecting the meninges and central nervous system.

According to one aspect, the present invention provides novel drugs that are effective, unlike currently available drugs, after direct administration (e.g., intrathecal) into barrier-protected space filled with cerebrospinal fluid (CSF). One shortcoming with conventional drugs is that upon being delivered to CSF, they do not remain there and leave the compartment before exerting the desirable therapeutic action. Only a very limited number of agents (mostly ligands of neurotransmitters) have been effective after intrathecal delivery (mostly as short term anesthetics).

In certain embodiments, the present invention provides methods for delivering therapeutic drugs and/or diagnostic agents selectively and for a prolonged period of time (several hours to days) to target tissues by a single injection. It will be appreciated that such methods can enable high local therapeutic efficiency in target areas without systemic side effects. In some embodiments, provided methods allow for administration of a broad range of drug substances that are otherwise unsuitable due to poor solubility. While not wishing to be bound by any particular theory, it is believed that the provided methods may afford greater safety and efficacy of administration, thereby decreasing morbidity and mortality, improving life expectancy, increasing cure rate, and reducing the cost of drug administration.

In certain embodiments, the present invention provides methods comprising the step of administering to an animal suffering from or susceptible to a cerebral, meningeal or neural disease, disorder, or condition a conjugate comprising a carrier substituted with one or more occurrences of a moiety having the structure:

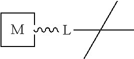

wherein:
each occurrence of M is independently a modifier;
[symbol] denotes direct or indirect attachment of M to linker L;
each occurrence of L is independently a linker; and
L is directly or indirectly attached to the carrier;
wherein the conjugate is administered directly into the cerebrospinal fluid space of the animal.

In certain embodiments, the present invention provides methods comprising the step of administering to an animal suffering from or susceptible to an infection or infectious disease of the brain or CSF space a conjugate comprising a carrier substituted with one or more occurrences of a moiety having the structure:

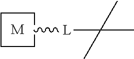

wherein:
each occurrence of M is independently a modifier;
[symbol] denotes direct or indirect attachment of M to linker L;
each occurrence of L is independently a linker; and
L is directly or indirectly attached to the carrier;
wherein the conjugate is administered directly into the cerebrospinal fluid space of the patient.

In some embodiments, the present invention provides methods comprising the step of administering to an animal suffering from or susceptible to a meningeal or neural disorder a conjugate comprising a carrier substituted with one or more occurrences of a moiety having the structure:

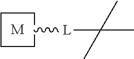

wherein:
each occurrence of M is independently a modifier;
[symbol] denotes direct or indirect attachment of M to linker L;
each occurrence of L is independently a linker; and
L is directly or indirectly attached to the carrier;
wherein the conjugate diffuses into the cerebrospinal fluid space of the animal via disease or injury-disrupted BBB.

In some embodiments, the conjugate is administered directly into the cerebrospinal fluid space of the animal. In some embodiments, the animal is a human.

In some embodiments, the present invention provides such methods of administration as described above, wherein the disease, disorder, or condition is a tumor of the brain or metastases of other primary tumors to the brain. In some embodiments, the disease, disorder, or condition is selected from the group consisting of neoplastic meningitis, meningiomas, Alzheimer disease, geriatric conditions, neuropathies, lysosomal storage diseases, pain, and pernicious anemia.

### Carriers

Any carrier, as defined above, is suitable for use with methods of the present invention. One of ordinary skill in the art is familiar with suitable carriers and characteristics thereof. It will be appreciated that a suitable carrier will comprise one or more functional groups for attaching a modifier and/or linker.

In some embodiments, the size (molecular weight) of the carrier molecule is selected such as to prevent fast clearance of the conjugate from CSF. The desired size of the carrier, as expressed in Daltons or length units (nanometers) generally depends on the size of other components in the conjugate. One of ordinary skill will be knowledgeable of various methods that can be used to determine carrier (particle) size. Such methods include size exclusion chromatography, dynamic light scattering, and transmission electron microscopy. In some embodiments, the carrier size is selected such that the total diameter of the conjugate is >2 nm. In some embodiments, the total diameter is >5 nm, >10 nm, >20 nm, >30 nm, or >50 nm. In some embodiments, the particle size or the conjugate is between 5 nm and 10 nm, between 5 nm and 20 nm, between 10 nm and 30 nm, between 10 nm and 50 nm, or between 20 nm and 50 nm.

In some embodiments, the size of the carrier is >1 kDa. In some embodiments, the size of the carrier is >2 kDa. In some embodiments, the size of the carrier is >3 kDa. In some embodiments, the size of the carrier is >4 kDa. In some embodiments, the size of the carrier is >5 kDa. In some embodiments, the size of the carrier is >6 kDa. In some embodiments, the size of the carrier is >7 kDa. In some embodiments, the size of the carrier is >8 kDa. In some embodiments, the size of the carrier is >9 kDa. In some embodiments, the size of the carrier is >10 kDa.

Without wishing to be bound by any particular theory, it is believed that, irrespective of the size of a modifier, the increased size afforded by conjugation with a carrier is useful in prolonging a conjugate's time of residence in the CSF. Accordingly, it will be understood that macromolecular modifiers may be smaller in size than their conjugated derivatives.

In some embodiments, e.g. where the drug is an enzyme, a carrier molecule prolongs drug activity in the lysosomal environment by slowing drug inactivation by lysosomal enzymes and/or the products of such enzymatic activity. One skilled in the art can select or develop such carriers by testing of various carrier and linker structures under lysosomal conditions in vitro, e.g., in a lysosomal extract.

In some embodiments, a carrier is water-soluble. In some embodiments, a carrier is nontoxic. In some embodiments, a carrier is nonimmunogenic. In some embodiments, a carrier is polymeric. In some embodiments, a carrier is biodegradable. Examples of suitable carriers are described by Haag, R. et al., Angew. Chem. Int. Ed. 2006, 45, 1198-1215; Ganta, S. et al. J. Controlled Release. 2008, 126, 187-204; and Slayton, P. S. et al. Multifunctional Pharmaceutical Nanocarriers, V. Torchilin (ed.) 2008, 143-159, the contents of each of which are hereby incorporated by reference.

Examples of hydrophilic biocompatible carriers include, without limitation, polyethylene glycol, HPMA, polyvinyl alcohol, water-soluble polyacrylates, polyoxazolines, polyamidoamines, polyals, plasma proteins, dextrans, polydextrins, water-soluble polyesters, polyamides, and other water-soluble polymers.

In certain embodiments, methods of the present invention employ conjugates comprising a biodegradable polymer carrier. Biodegradability is typically accomplished by synthesizing or using polymers that have hydrolytically unstable linkages in the backbone. The most common chemical backbone components with this characteristic are esters and amides. Novel polymers have been developed with anhydride, orthoester, polyacetal, polyketal and other biodegradable backbone components. Hydrolysis of the backbone structure is the prevailing mechanism for the degradation of such polymers. Other polymer types, such as polyethers, may degrade through intra- or extracellular oxidation. Biodegradable polymers can be either natural or synthetic. Synthetic polymers commonly used in medical applications and biomedical research include polyethyleneglycol (pharmacokinetics and immune response modifier), polyvinyl alcohol (drug carrier), and poly(hydroxypropylmetacrylamide) (drug carrier). In addition, natural polymers are also used in biomedical applications. For instance, dextran, hydroxyethylstarch, albumin, polyaminoacids and partially hydrolyzed proteins find use in applications ranging from plasma expanders, to radiopharmaceuticals to parenteral nutrition. In general, synthetic polymers may offer greater advantages than natural materials in that they can be tailored to give a wider range of properties and more predictable lot-to-lot uniformity than can materials from most natural sources. Methods of preparing various polymeric materials are well known in the art. In many biomedical applications, polymer molecules should be chemically associated with the drug substance, or other modifiers, or with each other (e.g., forming a gel). Several properties of the final product depend on the character of association, for example, drug release profile, immunotoxicity, immunogenicity and pharmacokinetics. In certain embodiments, provided methods afford drug release in under physiological conditions with an optimal rate and in a chemical form or forms optimally suited for the intended application.

In certain embodiments, the conjugates of the invention find use in biomedical applications, and the carrier is biocompatible and biodegradable. In certain embodiments, the carrier is a macromolecule, a molecular matrix (e.g., a gel or a solid) or an interface. In certain embodiments, the carrier is a macromolecule, soluble polymer, nanoparticle, gel, liposome, micelle, suture, implant, etc. In certain embodiments, the term “soluble polymer” encompasses biodegradable biocompatible polymer such as a polyal (e.g., hydrophilic polyacetal or polyketal). In certain embodiments, a carrier is a fully synthetic, semi-synthetic or naturally-occurring polymer. In certain embodiments, a carrier is hydrophilic.

In certain embodiments, carriers used in the present invention are biodegradable biocompatible polyals comprising at least one hydrolyzable bond in each monomer unit positioned within the main chain. This ensures that degradation processes (via hydrolysis/cleavage of the monomer units) will result in fragmentation of the polymer conjugate to the monomeric components (i.e., degradation), and confers to the polymer conjugates of the invention their biodegradable properties. The properties (e.g., solubility, bioadhesivity and hydrophilicity) of biodegradable biocompatible polymer conjugates can be modified by subsequent substitution of additional hydrophilic or hydrophobic groups.

Examples of biodegradable biocompatible polymers suitable for practicing the invention can be found inter alia in U.S. Pat. Nos. 5,811,510; 5,863,990 and 5,958,398; European Patent Nos.: 0820473 and 03707375.6; U.S. Patent Application Publication Nos. 2006/0069230 and 2007/0190018; and International Application Publication Nos. WO/2003/059988, WO/2004/009082, and WO/2005/023294, each of the above listed patent documents is incorporated herein by reference in its entirety. Guidance on the significance, preparation, and applications of this type of polymers may be found in the above-cited documents. In certain embodiments, the present invention will be useful employing carriers and complexes as described in U.S. Patent Application Publication No.: 2006/0019911, the entire contents of which are incorporated herein by reference.

In certain embodiments, biodegradable biocompatible polymer carriers, used for preparation of polymer conjugates of the invention, are naturally occurring polysaccharides, glycopolysaccharides, synthetic polymers of polyglycoside, polyacetal, polyamide, polyether, and polyester origin, or products of their oxidation, fuctionalization, modification, cross-linking, and conjugation.

In certain embodiments, a carrier is a hydrophilic biodegradable polymer selected from the group consisting of carbohydrates, glycopolysaccharides, glycolipids, glycoconjugates, polyacetals, polyketals, and derivatives thereof.

In certain embodiments, a carrier is a naturally occurring linear and branched biodegradable biocompatible homopolysaccharide selected from the group consisting of cellulose, amylose, dextran, levan, fucoidan, carraginan, inulin, pectin, amylopectin, glycogen and lixenan.

In certain embodiments, a carrier is a naturally occurring linear and branched biodegradable biocompatible heteropolysaccharide selected from the group consisting of agarose, hyluronan, chondroitinsulfate, dermatansulfate, keratansulfate, alginic acid and heparin.

In certain embodiments, a carrier is not a lipid. In certain embodiments, the carrier does not comprise phospholipids. In certain embodiments, the carrier does not comprise triglycerides. In certain embodiments, the carrier does not comprise cholesterol. In some embodiments, the carrier is not a liposome.

In some embodiments, a carrier is a hydrophilic polymer selected from the group consisting of polyacrylates, polyvinyl polymers, polyesters, polyorthoesters, polyamides, polypeptides, and derivatives thereof.

In certain embodiments, a carrier comprises polysaccharides activated by selective oxidation of cyclic vicinal diols of 1,2-, 1,4-, 1,6-, and 2,6-pyranosides, and 1,2-, 1,5-, 1,6-furanosides, or by oxidation of lateral 6-hydroxy and 5,6-diol containing polysaccharides prior to conjugation with one or more modifiers.

In some embodiments, carriers of the invention comprise activated hydrophilic biodegradable biocompatible polymer carriers comprising from 0.1% to 100% polyacetal moieties represented by the following chemical structure:

(—O—CH₂—CHR₁—O—CHR₂—)ₙ

wherein R₁ and R₂ are independently hydrogen, hydroxyl, carbonyl, carbonyl-containing substituent, a biocompatible organic moiety comprising one or more heteroatoms or a protected hydrophilic functional group; and n is an integer from 1-5000. In some embodiments, n is from 10-5000. In some embodiments, n is from 100-5000. In some embodiments, n is from 10-5000. In some embodiments, n is from 500-5000. In some embodiments, n is from 10-2500. In some embodiments, n is from 10-1000.

In some embodiments, the present invention provides methods as described above, wherein the carrier is a polyacetal. In certain embodiments, at least a subset of the polyacetal repeat structural units have the following chemical structure:

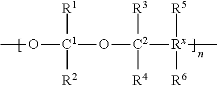

wherein for each occurrence of the n bracketed structure, one of R¹ and R² is hydrogen, and the other is a biocompatible group and contains a carbon atom covalently attached to C¹; R^{x} is a carbon atom covalently attached to C²; n is an integer; each occurrence of R³, R⁴, R⁵ and R⁶ is a biocompatible group and is independently hydrogen or an organic moiety; and for each occurrence of the bracketed structure n, at least one of R¹, R², R³, R⁴, R⁵ and R⁶ comprises a functional group suitable for coupling with a succinamide through an ester bond. In certain embodiments, n is an integer from 1-5000. In some embodiments, n is from 10-5000. In some embodiments, n is from 100-5000. In some embodiments, n is from 10-5000. In some embodiments, n is from 500-5000. In some embodiments, n is from 10-2500. In some embodiments, n is from 10-1000.

In certain embodiments, the present invention provides methods as described above, wherein the carrier is a polyketal. In certain embodiments, at least a subset of the polyketal repeat structural units have the following chemical structure:

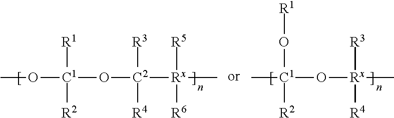

wherein each occurrence of R¹ and R² is a biocompatible group and contains a carbon atom covalently attached to C¹ or OC¹; R^{x} is a carbon atom covalently attached to C² or OC¹; n is an integer; each occurrence of R³, R⁴, R⁵ and R⁶ is a biocompatible group and is independently hydrogen or an organic moiety; and for each occurrence of the bracketed structure n, at least one of R¹, R², R³, R⁴, R⁵ and R⁶ comprises a functional group suitable for coupling with a succinamide through an ester bond. In certain embodiments, n is an integer from 1-5000. In some embodiments, n is from 10-5000. In some embodiments, n is from 100-5000. In some embodiments, n is from 10-5000. In some embodiments, n is from 500-5000. In some embodiments, n is from 10-2500. In some embodiments, n is from 10-1000.

In some embodiments, a carrier is PHF. PHF, or poly-[hydroxymethylethylene hydroxymethylformal] is a long-circulating, biocompatible, non-toxic, hydrophilic polymer (polyacetal) developed as an acyclic mimetic of interface polysaccharides universally present on cell surfaces (Papisov M I. Adv. Drug Delivery Rev. 1998, 32:119-138). PHF can be represented by a variety of chemical structures, depending on repeating unit, end groups, etc. One representation is given in Scheme 1, below.

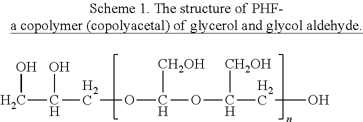

PHF is a highly hydrophilic, water soluble polymer, stable in physiological conditions, but undergoing proton-catalyzed hydrolysis at lysosomal pH. The polymer shows no toxicity in mice at doses up to 4 g/kg IV and IP (higher doses not studied). Upon IV administration, low molecular weight PHF (<50 kDa) is almost completely cleared by kidneys with no significant accumulation in any tissues. High molecular weight PHF and derivatives (PHF modified macromolecules and model drug carriers) that are not cleared by kidneys circulate with half-lives up to 10-25 hours (rodents), with a nearly uniform final distribution (accumulation per g tissue in RES only twice as higher as in other organs). The latter suggests lack of recognition by phagocytes, other cells and recognition proteins (“stealth” properties) (Papisov M I. Adv. Drug Delivery Rev., Special issue on long circulating drugs and drug carriers, 1995, 16:127-137).

PHF has been prepared at large scale at a variety of molecular weights. The chemical structure of PHF enables a wide variety of modifications and derivatizations, via pendant OH groups and/or at least one terminal vicinal glycol group (A. Yurkovetskiy, S. Choi, A. Hiller, M. Yin, A. J. Fischman, M. I. Papisov. Biodegradable polyal carriers for protein modification. 29th Int. Symp. on Controlled Release of Bioactive Materials, 2002, Seoul, Korea. Controlled Release Society, Deerfield, Ill., 2002; paper #357). Several PHF derivatives have been synthesized and characterized as model biomedical preparations (protein and small molecule conjugates, gels, long-circulating drug carriers, etc.) (Papisov M I. ACS Symposium Series 786 (2001), 301-314; Papisov M I, Babich J W, Dotto P, Barzana M, Hillier S, Graham-Coco W, Fischman A J. (1998) 25th Int. Symp. on Controlled Release of Bioactive Materials, 1998, Las Vegas, Nev., USA; Controlled Release Society, Deerfield, Ill., 170-171; Yurkovetskiy A, Choi S, Hiller A, Yin M, McCusker C, Syed S, Fischman A J, and Papisov M. Biomacromolecules 2005, 6:2648-2658; U.S. Pat. Nos. 5,811,510; 5,863,990 and 5,958,398; U.S. Patent Application Publication Nos. 2006/0019911, 2006/0069230, and 2007/0190018; and International Application Publication Nos. WO/2003/059988, WO/2004/009082, and WO/2005/023294).

Due to the “stealth” properties, biodegradability profile, and technological flexibility, PHF is a highly promising material for several pharmaceutical and bioengineering applications. In particular, the biodegradability and multifunctionality of PHF eliminates several limitations on the size and structure of small molecule conjugates, enabling, for example, high dose administration of high molecular weight conjugates (>50 kDa) without the risk of long-term polymer depositions in cells. In Applicant's own studies (Papisov, A et al. Hydrophilic Polyals: Biomimetic Biodegradable Stealth Materials for Pharmacology and Bioengineering. Proceedings of 226th Natl. Meeting of American Chemical Society, New York, N.Y.) as well as in studies conducted using Applicant's materials (U.S. Pat. No. 7,160,924), protein conjugates of PHF showed no renal cell vacuolization—in contrast with analogous PEG conjugates administered at the same doses. This result suggests that the safety concerns associated with high dose polymer administration (as in antineoplastic formulations requiring, in most cases, administration of hundreds of mg/kg), can be more effectively addressed using biodegradable “stealth” polymers such as PHF.

In certain embodiments, a carrier is a linear macromolecule, a branched macromolecule, a globular macromolecule, a graft copolymer, a comb copolymer, a nanoparticle, or a lipid-based carrier. In certain embodiments, a lipid-based carrier is a liposome.

In some embodiments, a carrier is a hydrophilic macromolecule that releases a hydrophobic drug.

Biodegradable biocompatible conjugates can be prepared to meet desired requirements of biodegradability and hydrophilicity. For example, under physiological conditions, a balance between biodegradability and stability can be reached. For instance, it is known that macromolecules with molecular weights beyond a certain threshold (generally, above 50-100 kDa, depending on the physical shape of the molecule) are not excreted through kidneys, as small molecules are, and can be cleared from the body only through uptake by cells and degradation in intracellular compartments, most notably lysosomes. This observation exemplifies how functionally stable yet biodegradable materials may be designed by modulating their stability under general physiological conditions (pH=7.5±0.5) and at lysosomal pH (pH near 5). For example, hydrolysis of acetal and ketal groups is known to be catalyzed by acids, therefore polyals will be in general less stable in acidic lysosomal environment than, for example, in blood plasma. One can design a test to compare polymer degradation profile at, for example, pH=5 and pH=7.5 at 37° C. in aqueous media, and thus to determine the expected balance of polymer stability in normal physiological environment and in the “digestive” lysosomal compartment after uptake by cells. Polymer integrity in such tests can be measured, for example, by size exclusion HPLC. One skilled on the art can select other suitable methods for studying various fragments of the degraded conjugates of this invention.

In many cases, it will be preferable that at pH=7.5 the effective size of the polymer will not detectably change over 1 to 7 days, and remain within 50% from the original for at least several weeks. At pH=5, on the other hand, the polymer should preferably detectably degrade over 1 to 5 days, and be completely transformed into low molecular weight fragments within a two-week to several-month time frame. Although faster degradation may be in some cases preferable, in general it may be more desirable that the polymer degrades in cells with the rate that does not exceed the rate of metabolization or excretion of polymer fragments by the cells. Accordingly, in certain embodiments, the conjugates of the present invention are expected to be biodegradable, in particular upon uptake by cells, and relatively “inert” in relation to biological systems. The products of carrier degradation are preferably uncharged and do not significantly shift the pH of the environment. It is proposed that the abundance of alcohol groups may provide low rate of polymer recognition by cell receptors, particularly of phagocytes. The polymer backbones of certain carriers (e.g., polyacetals and polyketals) utilized in methods of the present invention generally contain few, if any, antigenic determinants (characteristic, for example, for some polysaccharides and polypeptides) and generally do not comprise rigid structures capable of engaging in “key-and-lock” type interactions in vivo unless the latter are desirable. Thus, the soluble, crosslinked and solid conjugates of this invention are predicted to have low toxicity and bioadhesivity, which makes them suitable for several biomedical applications.

In certain embodiments, biodegradable biocompatible conjugates can form linear or branched structures. For example, biodegradable biocompatible polyal conjugates of the present invention can be chiral (optically active). Optionally, biodegradable biocompatible polyal conjugates of the present invention can be racemic.

In some embodiments, conjugates of the present invention are associated with a macromolecule or a nanoparticle. Examples of suitable macromolecules include, but are not limited to, enzymes, polypeptides, polylysine, proteins, lipids, polyelectrolytes, antibodies, ribonucleic and deoxyribonucleic acids, and lectins. A macromolecule may be chemically modified prior to being associated with said biodegradable biocompatible conjugate. Circular and linear DNA and RNA (e.g., plasmids) and supramolecular associates thereof, such as viral particles, for the purpose of this invention are considered to be macromolecules. In certain embodiments, conjugates of the invention are non-covalently associated with macromolecules.

In certain embodiments, conjugates of the invention are water-soluble. In certain embodiments, conjugates of the invention are water-insoluble. In certain embodiments, an inventive conjugate is in a solid form. In certain embodiments, conjugates of the invention are colloids. In certain embodiments, conjugates of the invention are in particle form. In certain embodiments, conjugates of the invention are in gel form. In certain embodiments, conjugates of the invention are in a fiber form. In certain embodiments, conjugates of the invention are in a film form.

### Modifiers

In certain embodiments, modifiers according to the invention include, but are not limited to, biomolecules, small molecules, organic or inorganic molecules, therapeutic agents, microparticles, pharmaceutically useful groups or entities, macromolecules, diagnostic labels, chelating agents, intercalator, hydrophilic moieties, dispersants, charge modifying agents, viscosity modifying agents, surfactants, coagulation agents and flocculants, to name a few. In certain embodiments, a modifier is a chemotherapeutic moiety. In some embodiments, a chemotherapeutic moiety is selected from the group consisting of alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel, epothilones, maytansinoids, tubulysins, aurora kinase inhibitors), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol).

In certain embodiments, a modifier is Taxol, which is optionally covalently bound to a secondary linker, and other non-limiting taxanes including taxotere. In certain embodiments, a modifier is camptothecin (CPT), which is optionally covalently bound to a secondary linker. In certain embodiments, non-natural CPT analogs might be employed. The term “non-natural CPT” means a compound based on the structure of the natural product camptothecin (CPT). Non-limiting examples of non-natural camptothecins include irinotecan, topotecan, SN-38, 9-aminocamptothecin, 9-nitrocamptothecin, edotecarin, rubitecan, gimatecan, namitecan, karenitecin, silatecan, lurtotecan, exatecan, diflomotecan, belotecan (CKD-602), GI-147211 (GG-211) and 539625. Other non-limiting examples for small molecules active against brain tumors or metastases when administered systemically include etoposide, vincristine, cyclophosphamide, lomustine, carmustine, BCNU, doxorubicin, procarbazine, platinum analogs (cisplatin, carboplatin), the nitrosureas, and temozolomide. All such small molecules are contemplated for use in accordance with the present invention.

In some embodiments, a modifier is other than cytarabine.

In some embodiments, a modifier is a biomolecule. Examples of biomolecules include, but are not limited to, enzymes, receptors, neurotransmitters, hormones, cytokines, cell response modifiers such as growth factors and chemotactic factors, antibodies, haptens, toxins, interferons, ribozymes, anti-sense agents, plasmids, DNA, and RNA.

In certain embodiments, a modifier is a small molecule. Examples of small molecules include, but are not limited to, drugs such as vitamins, anti-AIDS substances, anti-cancer substances, radionuclides, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics and imaging agents. One skilled in the art will select a drug in accordance with the biological activity that is desirable to achieve certain clinical objectives, for example eliminate or treat the cause or a symptom of a disease.

In certain embodiments, a modifer is a molecule having a molecular weight ≦about 10 kDa, ≦about 9 kDa, ≦about 8 kDa, ≦about 7 kDa, ≦about 6 kDa, ≦about 5 kDa, ≦about 4 kDa, ≦about 3 kDa, ≦about 2 kDa, or ≦about 1.5 kDa.

In some embodiments, modifier is characterized in that it immediately associates with the leptomeningeal compartment after the release from a carrier. In some embodiments, such association with the leptomeningeal compartment makes a modifer more resistant to washout from a target tissue.

Examples of suitable pharmaceutically useful groups or entities include, but are not limited to, hydrophilicity/hydrophobicity modifiers, pharmacokinetic modifiers, biologically active modifiers, and detectable modifiers.

In certain embodiments, a modifier may be chemically modified so that it comprises a functional group (i.e., amine group) suitable for covalent binding with an optionally substituted succinic acid through formation of an amide bond; said succinic acid being conjugated to a carrier through formation of an ester bond (vide infra).

In some embodiments, a modifier is released from a conjugate in a form that is active or convertible to an active form either spontaneously or by enzymes present in the CSF and/or in tissues receiving a modifier from the CSF. In some embodiments, a modifier is released in a highly hydrophobic form that forms long-term deposits in the CSF compartment or in the target tissues.

As described above, conjugates of the invention comprise one or more occurrences of M, where M is a modifier, wherein the one or more occurrences of M may be the same or different. In certain embodiments, one or more occurrences of M is a biocompatible moiety. In certain embodiments, one or more occurrences of M is a hydrophilic moiety. In certain embodiments, one or more occurrences of M is a drug molecule. In certain embodiments, one or more occurrences of M is a chemotherapeutic moiety. In some embodiments, one or more occurrences of M is a drug effective against cancer. In certain embodiments, one or more occurrences of M is a camptothecin moiety. In certain embodiments, one or more occurrences of M is a single- or double stranded oligonucleotide or polynucleotide. In certain embodiments, one or more occurrences of M is a peptide or protein. In certain embodiments, one or more occurrences of M is an siRNA molecule.

In some embodiments, a modifier is released from a carrier prior to the modifier exerting a desired biological effect. In some embodiments, a drug or prodrug modifier released from a carrier is selected from a group of substances with similar biological activities such that the properties of the released substance facilitate its access to a target tissue and/or cell. Such considerations will depend on the character of the target and its location in the tissues with respect to CSF. In certain embodiments, a released modifier has a molecular weight greater than about 20 kDa. In some embodiments, a released modifier is hydrophobic. In some embodiments, a released modifier is a hydrophobic prodrug (see Schemes 2 & 3, below). In certain embodiments, provided methods reduce the toxicity of a hydrophobic modifier by improving the solubility of the modifier.

In some embodiments, a target is located in the brain parenchyma and a released modifier has a relatively high molecular weight (e.g., greater than about 10 kDa, greater than about 12, greater than about 14 kDa, greater than about 16 kDa, or greater than about 18 kDa, greater than about 20 kDa, greater than 100 kDa). In some embodiments, the released modifier can be <2, >2, >5, >10. >20, >30 or >50 nm in hydrodynamic diameter.

In some embodiments, a target is located in the brain parenchyma or nerve tissue and a released modifier is hydrophobic.

In certain embodiments, a target benefits from or is amenable to very long drug action, and a released modifier is a hydrophobic prodrug.

In some embodiments, one or more occurrences of M is a hydrophobic drug. Non-limiting examples of hydrophobic drugs are taxoids, camptothecins, doxorubicin, michellamine B, vincristine, and cisplatin. The term “taxoid” is used to refer to paclitaxel, cephalomannine, baccatin III, 10-deacetyl baccatin III, deacetylpaclitaxel and deacetyl-7-epipaclitaxel and derivatives and precursors thereof. Paclitaxel is one example of a taxoid. Paclitaxel, also known as TAXOL™, (NSC 125973) is a diterpene plant product derived from the western yew Taxus brevifolia.

In certain embodiments, when the carrier is a polymer, about 2 to about 25% monomers comprise a modifier M. In certain embodiments, the percentage of monomers comprising a modifier M is about 5% to about 20%, about 5% to about 18%, about 5% to about 15%, about 6% to about 15%, about 6% to about 14%, about 7% to about 13%, about 7% to about 12%, about 8% to about 12%, about 9% to about 12%, about 10% to about 12%, about 9% to about 11%, or about 10% to about 11%.

In certain embodiments, a modifier comprises an amine functionality (or protected form thereof) which forms an amide bond upon reaction with the carboxylic acid group of a suitable succinic acid linker.

In certain embodiments, M is CPT. In certain embodiments, M is CPT and the secondary linker is an amino acyl residue. In certain embodiments, M is CPT and the secondary linker is a glycine residue.

In some embodiments, in conjugates of the invention, one or more occurrences of M comprises a biologically active modifier. In certain exemplary embodiments, one or more occurrence of M is selected from the group consisting of proteins, antibodies, antibody fragments, peptides, steroids, intercalators, drugs, hormones, cytokines, enzymes, enzyme substrates, receptor ligands, lipids, nucleotides, nucleosides, metal complexes, cations, anions, amines, heterocycles, heterocyclic amines, aromatic groups, aliphatic groups, intercalators, antibiotics, antigens, immunomodulators, and antiviral compounds. In certain embodiments, drugs include, but are not limited to, antineoplastic, antibacterial, antiviral, antifungal, antiparasital, anesthetic drugs.

In some embodiments, one or more occurrences of M is independently selected from the group consisting of biomolecules, small molecules, organic or inorganic molecules, therapeutic agents, detectable labels, microparticles, pharmaceutically useful groups or entities, macromolecules, DNA or RNA, anti-sense agents, gene vectors, virions, diagnostic labels, chelating agents, intercalator, hydrophilic moieties, dispersants, charge modifying agents, viscosity modifying agents, surfactants, coagulation agents and flocculants.

In certain embodiments, one or more occurrence of M comprises a detectable label. In certain exemplary embodiments, one or more occurrence of M comprises atoms or groups of atoms comprising radioactive, paramagnetic, superparamagnetic, fluorescent, or light absorbing structural domains.

In certain embodiments, one or more occurrences of M comprise a diagnostic label. Examples of diagnostic labels include, but are not limited to, diagnostic radiopharmaceutical or radioactive isotopes for gamma scintigraphy and PET, contrast agent for Magnetic Resonance Imaging (MRI) (for example paramagnetic atoms and superparamagnetic nanocrystals), contrast agent for computed tomography, contrast agent for X-ray imaging method, agent for ultrasound diagnostic method, agent for neutron activation, and moiety which can reflect, scatter or affect X-rays, ultrasounds, radiowaves and microwaves, fluorophores in various optical procedures, etc. Diagnostic radiopharmaceuticals include γ-emitting radionuclides, e.g., indium-111, technetium-99m and iodine-124, Cu-64, F-18, Ga-68, etc. Contrast agents for MRI (Magnetic Resonance Imaging) include magnetic compounds, e.g. paramagnetic ions, iron, manganese, gadolinium, lanthanides, organic paramagnetic moieties and superparamagnetic, ferromagnetic and antiferromagnetic compounds, e.g., iron oxide colloids, ferrite colloids, etc. Contrast agents for computed tomography and other X-ray based imaging methods include compounds absorbing X-rays, e.g., iodine, barium, etc. Contrast agents for ultrasound based methods include compounds which can absorb, reflect and scatter ultrasound waves, e.g., emulsions, crystals, gas bubbles, etc. In some embodiments, labels are substances useful for neutron activation, such as boron and gadolinium. Further, labels can be employed which can reflect, refract, scatter, or otherwise affect X-rays, ultrasound, radiowaves, microwaves and other rays useful in diagnostic procedures. Fluorescent or scattering labels can be used for photoimaging. In certain embodiments, a modifier comprises a paramagnetic ion or group.

In certain embodiments, a conjugate comprises a biologically active modifier and a detectable label.

In certain embodiments, a conjugate comprises a detectable label linked directly to the polymer chain.

In certain embodiments, a conjugate comprises a single- or double-stranded oligonucleotide linked covalently or non-covalently to one or more carrier molecules.

In certain embodiments, a conjugate comprises a linear or cyclic polynucleotide associated non-covalently with one or more carrier molecules.

### Linkers

Depending upon the type and structure of the target tissue, macromolecular conjugates described herein optionally contain linkers, tethers and/or other molecular domains that facilitate modifier delivery from the conjugate located in the CSF to the target tissue.

One skilled in the art will choose a linker that provides suitable modifier release profile with respect to the properties of the modifier, its biological activity, and the clinical objectives to be achieved by the administration of the conjugate.

In certain embodiments, linkers are groups of covalently associated atoms. An example of such a linker is an aliphatic tether containing a hydrolyzable bond, such as, without limitation, an amide, ester, acetal, ketal, oxime bond. Another example is a linker containing an oxidizable or reducible bond, such as, without limitation, a dithiol (—S—S—) bond.

In certain embodiments, linkers comprise non-covalently associated moieties or groups of atoms belonging to two different molecules. An example of the latter is a hybridized sequence of two partially complementary oligonucleotide chains.

In certain embodiments, linkers contain photosensitive groups of atoms that facilitate modifier release upon exposure to light.

In certain embodiments, the modifier and carrier molecules separate (i.e., disassociate) from one another in the CSF. In certain embodiments, the modifier and carrier molecule separate (i.e., disassociate) from one another after conjugate uptake from the CSF by tissues or cells contacting the CSF.

In certain embodiments, linkers regulate the release of a modifier from the conjugate such that the modifier is well distributed in the CSF. In some embodiments, a biologically significant or major fraction of the released modifier reaches the target tissues well outside of the original point of the conjugate administration.

In certain embodiments, a linker is characterized in that it provides slow release of a modifier moiety from a conjugate, thus providing a continuous flow of active modifier molecules from CSF to a target tissue. One skilled in the art can observe the kinetics of modifier release in CSF under physiological conditions and the dynamics of the conjugate translocation in CSF and, from such data, select a release mechanism and calculate an optimal range of release rates, which can then be used for selecting a suitable linker.

In certain embodiments, a linker is non-enzymatically cleaved to release a modifier by a hydrolytic mechanism.

In certain embodiments, the kinetics of modifier release is of first order. In other embodiments, the kinetics of modifier release is non-first order.

In certain embodiments, the kinetics of modifier release can be characterized by half-release time under physiological conditions. In certain embodiments, where the release kinetics are of first order, the half-release time T_{R1/2}=ln(2)/k_{R}, where /k_{R} is the constant of the release rate as understood in chemical kinetics.

Modifier delivery to various tissues surrounding CSF may utilize linkers designed for different release rates. Although not wishing to be bound by any particular theory, it is possible that modifier delivery to tissues located near the administration site may require a shorter release rate than to tissues located far from the administration site.

In certain embodiments, the half release time T_{R1/2}>1 hour. In some embodiments, 0.1<T_{R1/2}<2 hours. In some embodiments, 0.1<T_{R1/2}<1 hours. In some embodiments, 0.5<T_{R1/2}<2 hours. In some embodiments, 1<T_{R1/2}<2 hours. In some embodiments, T_{R1/2}>2 hours. In some embodiments, T_{R1/2}>3 hours. In some embodiments, T_{R1/2}>4 hours. In some embodiments, T_{R1/2}>5 hours. In some embodiments, T_{R1/2}>6 hours. In some embodiments, T_{R1/2}>7 hours. In some embodiments, T_{R1/2}>8 hours. In some embodiments, T_{R1/2}>9 hours. In some embodiments, T_{R1/2}>10 hours.

In certain embodiments, a linker is characterized in that it degrades in the lysosomal compartment under the influence of low pH or enzymatic activity, thus providing release of active modifier moieties inside target cells upon conjugate uptake by such cells.

In certain embodiments, a linker is characterized in that it provides non-covalent sites for a modifier molecule to interact with a carrier, enabling a long-lasting equilibrium of conjugate-bound, free and target-bound forms.

In certain embodiments, a linker is characterized in that it comprises targeting moieties that bind or direct the conjugate to target cells, thus enhancing modifier delivery to target cells as compared to normal cells residing in CSF or in the surrounding tissues.

In some embodiments, a linker is characterized in that it releases the modifier into the cerebrospinal fluid at a rate sufficient to provide an efficient amount of the modifier.

In certain embodiments, where modifier separation from the carrier is not required to enable modifier action, a linker can be stable under physiological conditions. In certain embodiments, where the modifier is intended to act in a lysosomal environment after conjugate uptake by cells without release, a linker is stable under the lysosomal conditions.

In some embodiments, the present invention provides methods as described above, wherein each occurrence of L is independently comprises a moiety having the structure:

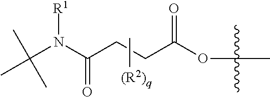

wherein

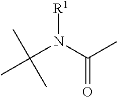

denotes the site of attachment to the modifier M;

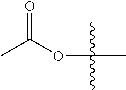

denotes the site of attachment to the carrier; p is an integer from 1-12; q is an integer from 0-4; R¹ is hydrogen, —C(═O)R^{1A}, —C(═O)OR^{1A}, —SR^{1A}, SO₂R^{1A} or an aliphatic, alicyclic, heteroaliphatic, heterocyclic, aryl, heteroaryl, aromatic, heteroaromatic moiety, wherein each occurrence of R^{1A} is independently hydrogen, alkyl, alkenyl, alkynyl, cycloalkyl, cycloalkenyl, cycloalkynyl, heteroalkyl, heteroalkenyl, heteroalkynyl, heterocycloalkyl, heterocycloalkenyl, heterocycloalkynyl, heteroaliphatic, heteroalicyclic, aromatic, heteroaromatic, aryl or heteroaryl; and each occurrence of R and R² is independently hydrogen, halogen, —CN, NO₂, an aliphatic, heteroaliphatic, aryl, heteroaryl, aromatic, heteroaromatic moiety, or -GR^{G1} wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC—, —NR—, —NR^{G2}SO₂—, —NR^{G2}SO₂NR^{G3}—, or —SO₂NR^{G2}—, wherein each occurrence of R^{G1}, R^{G2} and R^{G3} is independently hydrogen, halogen, or an optionally substituted aliphatic, heteroaliphatic, aromatic, heteroaromatic, aryl or heteroaryl moiety.

In some embodiments, the present invention provides methods as described above, wherein each occurrence of L is independently comprises a moiety having the structure:

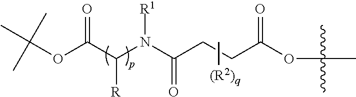

wherein

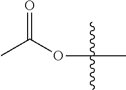

denotes the site of attachment to the modifier M;

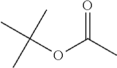

denotes the site of attachment to the carrier; p is an integer from 1-12; q is an integer from 0-4; R¹ is hydrogen, —C(═O)R^{1A}, —C(═O)OR^{1A}, —SR^{1A}, SO₂R^{1A} or an aliphatic, alicyclic, heteroaliphatic, heterocyclic, aryl, heteroaryl, aromatic, heteroaromatic moiety, wherein each occurrence of R^{1A} is independently hydrogen, alkyl, alkenyl, alkynyl, cycloalkyl, cycloalkenyl, cycloalkynyl, heteroalkyl, heteroalkenyl, heteroalkynyl, heterocycloalkyl, heterocycloalkenyl, heterocycloalkynyl, heteroaliphatic, heteroalicyclic, aromatic, heteroaromatic, aryl or heteroaryl; and each occurrence of R and R² is independently hydrogen, halogen, —CN, NO₂, an aliphatic, heteroaliphatic, aryl, heteroaryl, aromatic, heteroaromatic moiety, or -GR^{G1} wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC—, —NR—, —NR^{G2}SO₂—, —NR^{G2}SO₂NR^{G3}—, or —SO₂NR^{G2}—, wherein each occurrence of R^{G1}, R^{G2} and R^{G3} is independently hydrogen, halogen, or an optionally substituted aliphatic, heteroaliphatic, aromatic, heteroaromatic, aryl or heteroaryl moiety.

In some embodiments, q is 0. In some embodiments, p is 1.

In some embodiments, the present invention provides methods as described above, wherein each occurrence of L is independently comprises a moiety having the structure:

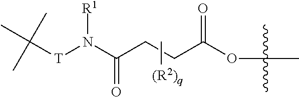

wherein:

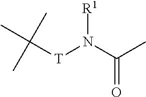

denotes the site of attachment to a modifier M;
T is a covalent bond or an optionally substituted, bivalent C₁₋₁₂ saturated or unsaturated, straight or branched, hydrocarbon chain, wherein one or more methylene units of L are independently replaced by -Cy-, —C(R^{x})₂—, NR^{x}—, —N(R^{x})C(O)—, —C(O)N(R^{x})—, —N(R^{x})SO₂—, —SO₂N(R^{x})—, —O—, —C(O)—, —OC(O)—, —C(O)O—, —S—, —SO—, —SO₂—, —C(═S)—, —C(═NR^{x})—, —N═N—, or —C(═N₂)—;
each Cy is independently an optionally substituted bivalent ring selected from phenylene, a 3-7 membered saturated or partially unsaturated carbocyclylene, a 3-7 membered saturated or partially unsaturated monocyclic heterocyclylene having 1-2 heteroatoms independently selected from nitrogen, oxygen, or sulfur, or a 5-6 membered heteroarylene having 1-3 heteroatoms independently selected from nitrogen, oxygen; and
   each R^{x} is independently hydrogen, a natural or unnatural amino acid side chain, or an optionally substituted group selected from C₁₋₆ aliphatic, phenyl, a 3-7 membered saturated or partially unsaturated carbocyclic ring, a 3-7 membered saturated or partially unsaturated monocyclic heterocyclic ring having 1-2 heteroatoms independently selected from nitrogen, oxygen, or sulfur, or a 5-6 membered heteroaryl ring having 1-3 heteroatoms independently selected from nitrogen, oxygen, or sulfur.

In certain embodiments, for any of the methods described above, the conjugate comprises a subset of L moieties on the carrier which are not substituted with a modifier M. In some embodiments, the unsubstituted sites have the structure:

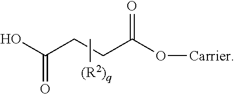

In certain embodiments, q is 0.

In certain embodiments, one or more occurrences of M is attached to a succinamide linker either directly or through a secondary linker. In certain embodiments, the secondary linker is an amino acyl residue, and the conjugate has the following general structure:

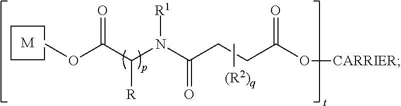

wherein p is an integer from 1-12; t is an integer designating the number of modifier moieties conjugated to the carrier; q is an integer from 0-4; R¹ is hydrogen, —C(═O)R^{1A}, —C(═O)OR^{1A}, —SR^{1A}, SO₂R^{1A} or an aliphatic, alicyclic, heteroaliphatic, heterocyclic, aryl, heteroaryl, aromatic, heteroaromatic moiety, wherein each occurrence of R^{1A} is independently hydrogen, alkyl, alkenyl, alkynyl, cycloalkyl, cycloalkenyl, cycloalkynyl, heteroalkyl, heteroalkenyl, heteroalkynyl, heterocycloalkyl, heterocycloalkenyl, heterocycloalkynyl, heteroaliphatic, heteroalicyclic, aromatic, heteroaromatic, aryl or heteroaryl; and each occurrence of R and R² is independently hydrogen, halogen, —CN, NO₂, an aliphatic, heteroaliphatic, aryl, heteroaryl, aromatic, heteroaromatic moiety, or -GR^{G1} wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC—, —NR—, —NR^{G2}SO₂—, —NR^{G2}SO₂NR^{G3}—, or —SO₂NR^{G2}—, wherein each occurrence of R^{G1}, R^{G2} and R^{G3} is independently hydrogen, halogen, or an optionally substituted aliphatic, heteroaliphatic, aromatic, heteroaromatic, aryl or heteroaryl moiety.

In some embodiments, p is 1. In some embodiments, p is 1 and R is hydrogen. In some embodiments, q is 0, p is 1, and R and R¹ are each hydrogen.

In certain embodiments, a secondary linker is an α-amino acyl residue, and the conjugate has the following general structure:

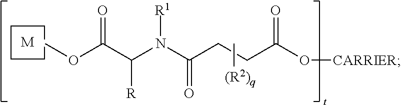

wherein t, R, R¹, R², and q are as defined above, and R designates a natural or unnatural amino acid side chain.

In some embodiments, the conjugate has the following general structure:

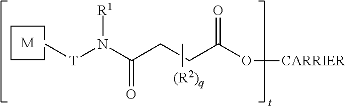

wherein:
T is a covalent bond or an optionally substituted, bivalent C₁₋₁₂ saturated or unsaturated, straight or branched, hydrocarbon chain, wherein one or more methylene units of L are independently replaced by -Cy-, —C(R^{x})₂—, NR^{x}—, —N(R^{x})C(O)—, —C(O)N(R^{x})—, —N(R^{x})SO₂—, —SO₂N(R^{x})—, —O—, —C(O)—, —OC(O)—, —C(O)O—, —S—, —SO—, —SO₂—, —C(═S)—, —C(═NR^{x})—, —N═N—, or —C(═N₂)—;
each Cy is independently an optionally substituted bivalent ring selected from phenylene, a 3-7 membered saturated or partially unsaturated carbocyclylene, a 3-7 membered saturated or partially unsaturated monocyclic heterocyclylene having 1-2 heteroatoms independently selected from nitrogen, oxygen, or sulfur, or a 5-6 membered heteroarylene having 1-3 heteroatoms independently selected from nitrogen, oxygen; and
each R^{x} is independently hydrogen, a natural or unnatural amino acid side chain, or an optionally substituted group selected from C₁₋₆ aliphatic, phenyl, a 3-7 membered saturated or partially unsaturated carbocyclic ring, a 3-7 membered saturated or partially unsaturated monocyclic heterocyclic ring having 1-2 heteroatoms independently selected from nitrogen, oxygen, or sulfur, or a 5-6 membered heteroaryl ring having 1-3 heteroatoms independently selected from nitrogen, oxygen, or sulfur.

In some embodiments, T is a bivalent C₁₋₆ saturated or unsaturated, straight or branched, hydrocarbon chain, wherein one, two, or three methylene units of L are independently replaced by —C(R^{x})₂—, NR^{x}—, —N(R^{x})C(O)—, —C(O)N(R^{x})—, —O—, or —C(O)—.

In some embodiments, when T is not a covalent bond, the atom of T connected to —NR¹— is a carbon atom.

### Conjugates

As explained above, in some embodiments, the present invention provides specialized conjugates for delivery to tissues contacting CSF. For any conjugate disclosed herein for use in methods of the present invention, compositions of such conjugates are contemplated as well. In some embodiments, a conjugate is a macromolecular conjugate. In certain embodiments, a conjugate comprises a modifier that is a drug. In certain embodiments, a conjugate comprising a drug is covalently associated with a macromolecular carrier molecule. In certain embodiments, a conjugate comprising a drug is non-covalently associated with a macromolecular carrier molecule. In some embodiments, the association of a modifier and carrier molecule is via a linker.

In certain embodiments, a conjugate is characterized in that it is capable of undergoing dual phase release based on a two-stage process of hydrolysis of esters of monosuccinamides. This reaction is mediated by a synchronous cyclization-elimination process (Scheme 2). The carrier macromolecule (e.g., PHF) is on the ester side, i.e., is connected with a drug-containing monosuccinamidate via an ester bond. Thus, the conjugate (I) first releases a succinamidate prodrug, (III) which subsequently releases (e.g., via hydrolysis) the active drug form (V).

Scheme 2. Dual stage drug release via cyclization-elimination in mono-succinamidate esters

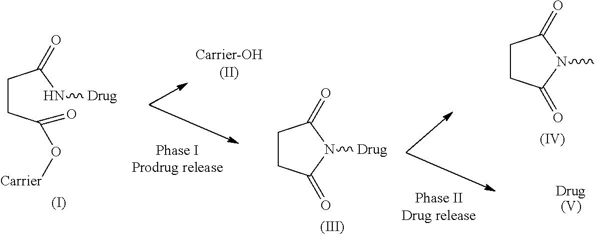

When the drug is camptothecin, the conjugate releases a highly hydrophobic prodrug form of camptothecin, CPT-SI (Scheme 3) (U.S. Patent Application Publication No. 2007/0190018; Yurkovetskiy A. V., Hiller A., Syed S., Yin M., Lu X. M., Fischman A. J., and Papisov M. I. Molecular Pharmaceutics 2004, 1:375-382). This prodrug remains at the release site and evenly (without focal extra- or intracellular deposition sites) distributes within the tissue. The lactone ring of CPT in the prodrug is stabilized and does not open until CPT is released in the free active form.

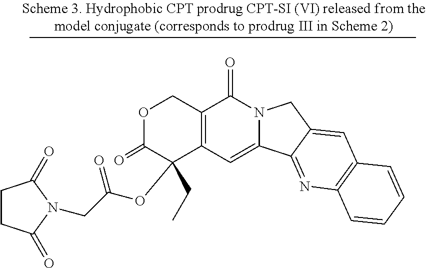

The mechanism of the second stage hydrolysis in vivo, which results in the active CPT release, may include spontaneous or enzymatic hydrolysis as well as, e.g., aminolysis. In vitro, the half-life of (N-succinamido)-glycyl linkage in the CPT prodrug (VI) in rat plasma was over 20 hours.

Treatment of two human cancer xenograft models in nude mice with CPT-PHF demonstrated that the combination of kinetic parameters of stage I and stage II release do enable a very efficient tumor growth suppression and longer survival at a twice weekly injection schedule. Thus, the dual stage CPT release model developed in Applicant's previous studies (is very well suitable for modeling intrathecal drug release. However, previously prepared conjugates were optimized for systemic administration, and thus the hydrodynamic molecular size of the conjugate may require modification for optimal CSF retention (see ensuing Examples).

In certain embodiments, a conjugate comprises one or more modifiers comprising an anchoring moiety. In certain embodiments, a conjugate comprises one or more anchoring protein molecules. In some embodiments, an anchoring protein molecule is an enzyme. In some embodiments, an anchoring protein is a human recombinant enzyme. In some embodiments, an anchoring moiety is a protein is selected from the group consisting of alpha or beta galactosidase, iduronate-2-sulfatase, idursulfase, arylsulfatase A, and sulfamidase.

In some embodiments, a PHF conjugate comprises an anchoring modifier. In some embodiments, a PHF conjugate comprises an anchoring protein. For example, in the synthesis of a PHF-succinamide-modifier conjugate, a significant number of unmodified carboxyl groups are present on the conjugate due to the fact that not all succinate residues are bonded to a modifer. These residues can be used to associate the conjugate with an anchoring protein via a carbodiimide-mediated reaction at pH 6.7, where both the protein and PHF, and modifier moieties are stable. The resultant PHF-modifier-protein conjugate is then purified by size exclusion chromatography and lyophilized.

In some embodiments, the present invention provides compositions comprising a conjugate, wherein the conjugate comprises a carrier substituted with one or more occurrences of a moiety having the structure:

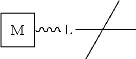

wherein:
each occurrence of M is independently a modifier;
[symbol] denotes direct or indirect attachment of M to linker L;
each occurrence of L is independently a linker; and
L is directly or indirectly attached to the carrier.

In some embodiments, one or more occurrences of M is independently a chemotherapeutic agent, a neuroprotective agent, an anti-infective agent, or a hydrophobic drug, with the proviso that M is not taxol or camptothecin. In certain embodiments, one or more occurrences of M is independently selected from the group consisting of mechlorethamine, chlorambucil, Cyclophosphamide, Melphalan, Ifosfamide, Methotrexate, 6-Mercaptopurine, BCNU, procarbazine, temozolomide, 5-Fluorouracil, Cytarabile, Gemcitabine, Vinblastine, Vincristine, Vinorelbine, Etoposide, Irinotecan, Topotecan, Doxorubicin, Bleomycin, Mitomycin, Carmustine, Lomustine, Cisplatin, Carboplatin, Asparaginase, Tamoxifen, Leuprolide, Flutamide, and Megestrol. In some embodiments, one or more occurrences of M is independently selected from the group consisting of irinotecan, topotecan, SN-38, 9-aminocamptothecin, 9-nitrocamptothecin, edotecarin, rubitecan, gimatecan, namitecan, karenitecin, silatecan, lurtotecan, exatecan, diflomotecan, belotecan (CKD-602), GI-147211 (GG-211) and 539625.

### Conjugate Administration

The brain is shielded against penetration of potentially harmful substances from the blood flowing through brain tissues by the blood-brain barrier (BBB). The brain capillary endothelium is much less permeable to solutes than other capillary endothelia. There is little transit across the BBB of large, hydrophilic molecules aside from some specific proteins such as transferrin, lactoferrin and low-density lipoproteins, which are taken up by receptor-mediated endocytosis (see Pardridge, J. Neurovirol. 5: 556-569 (1999)); Tsuji and Tamai, Adv. Drug Deliv. Rev. 36: 277-290 (1999); Kusuhara and Sugiyama, Drug Discov. Today 6:150-156 (2001); Dehouck, et al. J. Cell. Biol. 138: 877-889 (1997); Fillebeen, et al. J. Biol. Chem. 274: 7011-7017 (1999)).

On the outside boundaries, the brain, as well as the spinal cord, is surrounded by the CSF. The CSF is surrounded by meninges (arachnoid tissues, dura mater) which serve as a shield against penetration of potentially harmful substances from the outside tissues. The CSF is also shielded from blood flowing through the meninges by the blood-CSF barrier (The Blood-Cerebrospinal Fluid Barrier, by W. Zheng and A. Chodobski, Chapman & Hall/CRC, 2005).

The BBB and blood-CSF barriers present significant obstacles to drug delivery. Therefore, direct drug administration to the CSF has been attempted to treat brain and spinal cord diseases, or to regulate the spinal cord and/or large nerve functions. For the treatment of cancer specifically, it is believed that BBB can be disrupted allowing intravenous drug administration in a way that allows delivery to tumors.

Certain methods of drug administration into CSF are well known and include direct injections, implanted cranial and lumbar ports (in some cases equipped with various pumps), and image-guided techniques. In certain embodiments, provided methods utilize one of the known modalities. For example, drug injection into cisterna magna or lumbar injections into the CSF have been extensively studied in radiology and pain management. In some embodiments, chronic drug delivery to CSF in the lumbar area is achieved through implantation of lumbar ports. In certain embodiments, more invasive techniques are used, including drug delivery to brain ventricles through surgically implanted ports.

Due to the relatively rapid clearance of the injected drugs from the CSF, drug infusion through preinstalled ports is presently the only available way of maintaining therapeutic concentrations of drugs in the CSF (multiple injections over a short period of time compromise the integrity thus the barrier function of dura mater and other meninges). A one-time injection into the CSF in cisterna magna area is used almost exclusively for diagnostic purposes, where the injected preparation is required to stay in the CSF for the length of the diagnostic procedure. In certain embodiments, direct single injection in the lumbar area is used for short-term local or regional anesthesia (Spinal drug delivery, TL Yaksh, Elsevier, 1999).

Several models of leptomeningeal metastasis of various cancers in immunodeficient (nude) rats and mice were reported (Schabet M and Herrlinger U. J. Neurooncol. 1998, 38:199-205). The two established models of leptomeningeal breast cancer cell spread were both developed in nude rats (Bergman I, Ahdab-Barmada M, Kemp S S, Griffin J A and Cheung N-K V. Journal of Neuro-Oncology 1997, 34: 221-231; Bergman I, Barmada M A, Griffin J A and Slamon D J. Clinical Cancer Research 2001, 7:2050-56). Other non-breast cancer models were developed in nude mice. Rat models may be preferable to mouse models taking into account that at later stages rats provide a much better platform for extensive imaging studies (pharmacokinetics and tumor growth investigation). The breast cancer models utilize female athymic Rnu/nu rats and two human mammary adenocarcinoma cell lines, SK-BR-3 and MCF-7 (a.k.a. HTB-30 and HTB-22, respectively). Animals were implanted with 10⁸ cells each. SK-BR-3 cells are known HER2/neu-amplified/-overexpressing breast cancer cells, whereas MCF-7 express normal amounts of the receptor. SK-BR-3 cells showed strong subarachnoidal proliferation with nodular invasions in brain parenchyma. MCF-7 cells were less aggressive. The success rate with SK-BR-3 was 7/7 (100%), while in the MCF-7 group only 2 animals out of 8 showed progressive disease. Three animals out of 7 implanted with SK-BR-3 showed neurologic deficit (mean onset time 13.3 days); their mean day of death was 24. No animals implanted with MCF-7 demonstrated neurological deficit, and the mean day of death was 52. Both cell lines are sensitive to tecans (Kim D-K, Ryu D H, Lee J Y, Lee N, Kim Y-W, Kim J-S, Chang K, Im G-J, Kim T-K, and Choi W-S. J. Med. Chem., 2001, 44:1594-1602). By all parameters, the SK-BR-3 model is more suitable for drug efficacy investigation than MCF7, and can be used in accordance with the present invention.

The present invention encompasses the recognition that administration of drug (i.e., modifier) conjugates can provide greater efficacy and safety in the treatment of diseases and disorders of the meninges and central nervous system.

In certain embodiments, the present invention utilizes previously unknown properties of the biological compartment containing cerebrospinal fluid (CSF). According to one aspect, the present invention relates to Applicant's observation that large molecules (i.e., macromolecules) administered to the CSF, unlike small molecules, do not rapidly leave the CSF compartment. In addition, it has been found that such administered macromolecules spread relatively rapidly over the entire CSF volume, and that the compartment containing (or continuous with) CSF extends beyond the widely known ventricles, cisternae and cerebral and spinal arachnoids.

In certain embodiments, the present invention provides methods for administering a conjugate comprising a carrier substituted with one or more occurrences of a moiety having the structure:

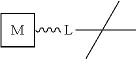

wherein:
each occurrence of M is independently a modifier;
[symbol] denotes direct or indirect attachment of M to linker L;
each occurrence of L is independently a linker; and
L is directly or indirectly attached to the carrier;
wherein the conjugate is administered directly into the cerebrospinal fluid space of an animal;
wherein the linker is characterized in that it releases the modifier into the cerebrospinal fluid space at a rate sufficient to provide an efficient amount of the modifier;
   wherein the conjugate displays continued residence in the cerebrospinal fluid for at least 30 minutes.

In some embodiments, a conjugate displays continued residence in the cerebrospinal fluid for at least 45 minutes, at least 1 hour, at least 1.5 hours, at least 2 hours, at least 3 hours, at least 4 hours, at least 5 hours, at least 6 hours, at least 8 hours, at least 10 hours, at least 12 hours, at least 16 hours, at least 20 hours, or at least 24 hours. In some embodiments, a conjugate displays continued residence in the cerebrospinal fluid for a time period between 30 min. and 2 hours, between 1 hour and 2 hours, between 1 hour and 4 hours, between 2 hours and 8 hours, between 4 hours and 12 hours, between 12 hours and 24 hours, or between 1 hours and 12 hours.

In certain embodiments of provided methods, the administration into the cerebrospinal fluid space is through intraventricular or lumbar port or direct intrathecal injection into cisterna magna or in the lumbar area. In certain embodiments, the administration if by infusion. In certain embodiments, the infusion is by an implated pump. In some embodiments, the intrathecal administration is by a ported catheter to the spinal fluid. In some embodiments, the administering introducing the conjugate into the lumbar area. In some embodiments, the administering introducing the conjugate into the cisterna magna.

As described in the ensuing examples, injection of macromolecules into the CSF compartment of animals, independently of macromolecule type, affords distribution of the macromolecule in the CSF compartment around the brain and spinal cord. Unexpectedly, it was found also that macromolecules are transported well beyond the meningeal and spinal compartments, apparently along major nerves.

In some embodiments, model proteins, independently of the protein type, rapidly (within seconds or minutes) distribute in the CSF compartment around the brain and spinal cord, and within a few hours can distribute over the entire CSF compartment regardless of the administration point. Unlike small molecules that leave the CSF compartment within minutes, the major fraction of large molecules remains in the CSF during the process of distribution in the CSF compartment.

Thus, unlike small molecules, macromolecules, being injected into CSF, do not clear from the CSF compartment within minutes but stay in CSF for a prolonged period of time. While not wishing to be bound by any particular theory, it appears that the boundaries of the CSF compartment have low permeability for large molecules, and this enables extended residence of large molecule modifiers in the CSF compartment. Thus, in some embodiments, conjugates comprise modifier molecules and macromolecular carriers, where the latter will hold the former in the CSF compartment for a prolonged period of time (measured in hours and days), thereby facilitating modifier access to the target tissues and cells.

The CSF-containing compartment borders with a variety of tissues that have various macro- and micro-structural organization. Pathologies may further modify the normal structure and function of the tissues (e.g., inflammation) or introduce new, pathological tissues that differ in structure and function with the normal ones (e.g., cancer). The “structure” in this context includes, but is not limited to, morphology, types of cells, surface markers expressed on cell surfaces, composition and morphology of the extracellular matrix, etc. The “functions” include, but are not limited to, transport of liquid and solutes, binding, endocytosis, enzymatic cleavage, etc. Pathological cells or pathogens, such as bacteria, fungi, parasites, viruses, may also be present in the CSF. In some embodiments, the present invention provides methods for treatment of an infectious disease where the modifier is delivered to the CSF or to the surrounding normal or pathological tissues.

Provided methods are also useful for diagnostic purposes and/or for evaluation of the efficacy of therapy. In some embodiments, a detectable label is incorporated into the structure of a conjugate along with, or instead of, a drug or prodrug moiety. Diagnostic embodiments of the present invention can be used, for example, for investigation of the continuity of the CSF compartment, pathological changes in the CSF or the surrounding tissues, or enzyme activity in the CSF and/or in the tissues contacting the CSF, or for investigation of the drug distribution in and around the CSF compartment.

In certain embodiments, the step of detecting the detectable modifier is performed non-invasively. In certain exemplary embodiments, the step of detecting the detectable modifier is performed using suitable imaging equipment.

The CSF surrounds the brain and spinal cord, and CSF is mechanically “pumped” around the brain by the brain and spinal cord tissues oscillating due to the arterial pulsation. It is known that CSF is constantly replaced via (1) production of new fluid by the choroids plexuses in the brain ventricles, and (2) absorption and transport of the fluid to venous blood in the arachnoid.

While not wishing to be bound by any particular theory, it is believed that the CSF may also drain into extrameningeal interstitial space (and subsequently into the lymphatic system) at unknown sites. Ex-vivo animal experiments on filling the CSF compartment under high pressure with a liquid silicone compound have shown that the silicone can move along large nerves. It is unknown whether silicone penetrates along the nerves by damaging them and physically opening new channels in the damaged tissues or such channels do exist in normal undamaged nerves.

In certain embodiments, the efficacy of a conjugate delivery to a target within the CSF compartment is further enhanced by associating the conjugate with a molecular or supramolecular entity that can be physically guided through the CSF by an action of an externally applied field. In some embodiments, the externally applied field is gravitational. In some embodiments, the externally applied field is magnetic. In some embodiments, the externally applied field is electromagnetic.

It is known, for example, that addition of a substance that makes a solution heavier than the surrounding liquid will make a drop or a slow stream of such solution move downwards. Thus, it will be appreciated that a patient can be positioned in such a way that a “heavy” solution of a conjugate will translocate from the injection point (e.g., cisterna magna) to the target site. (Yaksh, supra).

Addition of a magnetic colloid (e.g., superparamagnetic nanoparticles) to a solution—even at low concentrations—results in a liquid that can be magnetically guided through biological liquids and held in place, for a period of time, in a flow of biological liquid. Magnetized liquids translocate along the magnetic field gradients (Rusetsky A N, Papisov M I, Ruuge E K, Torchilin V P. Substantiation of using magnet-directed localization of drugs for the treatment of thrombosis (Rus., Engl. abstract). Bull. USSR Cardiol. Res. Center 1985; 1:100-5). The present invention contemplates methods comprising a magnet or a system thereof that guide a drug to a target, even against the flow of CSF, almost without dilution. Even locations presently inaccessible for drugs administered to the CSF, such as central ventricle, could be accessible through such methods. Non-toxic magnetic colloids allowed for human use are commercially available (e.g., Ferridex), and a system of magnets (or a single magnet) can be engineered to guide the drug through CSF. A magnetic guidance system enables patient treatment in any position (e.g., sitting), which can be preferable to gravitational guidance.

In certain embodiments, the action of the conjugates is further modulated by associating them with molecular or supramolecular entities enabling conjugate binding to the target tissue. The examples of such entities include, without limitation, antibodies, receptor ligands, cationic moieties, oligo- and polysaccharides, proteins, peptides, oligonucleotides. One skilled in the art can select or develop such entities either based on knowledge of the structure of the target tissue or cells, or by selection from suitable libraries, e.g., phage display or oligonucleotide libraries, through testing such libraries against the target tissues or cells.

In some embodiments, the action of the conjugates is further modulated by the physical guidance of the drug from the injection site to the known site of disease, for example, by (electro)magnetic field or gravitation.

### Uses

Neoplastic meningitis is a devastating complication of breast cancer and other solid tumors (Jayson G. C. & Howell A. Annals of Oncology 1996, 7: 773-786). Published data suggest that 5-8% of patients suffer from this complication (Bleyer W A. Curr Probl Cancer 1988; 12:185-237). About 5% of the patients have metastatic meningitis on the first relapse, and 20% on the second. Autopsy data suggest a prevalence of 20% in patience who die as a result of systemic cancer (Posner J B. Neurologic Complications of Cancer. Philadelphia, F.A. Davis Co., 1995, p 143). The most common neurological features include headaches, abnormalities of cranial nerve function (mostly the nerves supplying the muscles of extra-ocular movement and the facial nerves) and cerebral symptoms such as speech disturbance. Spinal symptoms such as root pain and dysfunction producing limb weakness, are also common. Any of these features can exist together, producing a mononeuritis multiplex.

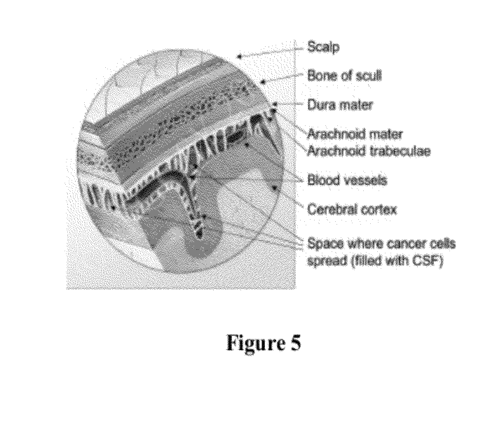

Neoplastic meningitis develops in the space enveloped between the brain and the external meningeal sheath (dura mater) that fully surrounds the brain (FIG. 5). The space is filled with CSF. Seeding of the leptomeninges, the arachnoid and pia mater, by cancer cells causes neoplastic meningitis. The microscopic findings include sheets of cancer cells, with accompanying local fibrosis, that line the meninges, ensheathing blood vessels and nerves (DiChiro G, Hammock M K, Bleyer W A. Neurology 1976; 26:1-8; Grossman S A, Moynihan T J. Neurol Clin 1991; 9:843-56).

The current treatment is largely palliative (Pace A, Fabi A. Crit. Rev Oncol Hematol. 2006, 60:194-200). Three modalities have been used: craniospinal irradiation, systemic chemotherapy (with chemotherapy and/or glucocorticoids), and local chemotherapy via lumbar puncture or intraventricular (Ommaya) reservoir. It was found that systemically administered therapeutics do not reach cancer cells residing in the meninges. The leptomeningeal space is well isolated from the rest of the body by the blood-brain barrier (BBB) on one side, and by the blood-CSF barrier (The blood-Cerebrospinal Fluid Barrier. Zheng W. and Chodobski A., eds. Taylor & Francis 2004, Boca Raton, Fla.) on the other, which makes systemic chemotherapy ineffective. For example, the CSF level of systemically administered methotrexate is only 1.7% of the systemic level. Attempts have been made to treat neoplastic meningitis with drugs administered directly to the cerebro-spinal fluid (Chamberlain M C, Tsao-Wei D D, and Groshen S. CANCER 2006, 106:2021-7). However, the currently available chemotherapeutics are rapidly cleared from CSF, and even continuous administration or liposomal formulations did not result in significant improvements. The median survival of breast cancer patients with neoplastic meningitis is about 3 months (Jaeckle K A. Semin Oncol. 2006, 33:312-23; Jayson G. C. &Howell A. Annals of Oncology 1996, 7: 773-786) and only 2-4 weeks (Boogerd W & Hart A A M. Cancer 1991, 67:1685-95) if not aggressively treated.

Although the location, volume and the mechanism of the formation of CSF are well studied, the details of CSF translocation within and, especially, from the leptomeningeal compartment are very poorly understood. The clearance of solutes from the leptomeningeal compartment also has not been systematically studied. The current view is that CSF predominantly drains from the leptomeningeal compartment directly into blood and/or lymphatic vessels, without any filtration. Nevertheless, the rate of macromolecule clearance from CSF to the system appears to decrease with the molecule size. Still on the other hand, some reported protein clearance data suggest that an (unknown) protein-specific mechanism of active clearance may exist in the leptomeninges. Thus, and without wishing to be bound by any particular theory, macromolecules of non-protein nature that don't interact with any protein-recognizing molecular mechanisms may be better suited as intrathecal drug carriers than proteins. The behavior of such molecules in CSF (in particular, size dependence of transfer and clearance processes) has not been studied.

The published data suggest that the bulk of the CSF is produced by the choroid plexuses in the brain ventricles. Some (unknown) volume is added as the interstitial fluid from the brain parenchyma mixes with the plexus-secreted CSF. There is no evidence of CSF formation in the spinal region, and the prevalent flow of CSF is considered to be anteriodorsal, from the intracranial compartments to the spinal ones. The flow is locally perturbed by the pulsatile movement of the brain and meningeal tissues.

Several studies have been attempted to determine the locations and mechanisms of CSF drainage to the outside of the leptomeningeal space. It has been proposed that the fluid may drain either into interstitium outside CNS at various locations and then into the lymphatic tissues, while another fraction may be drained straight into the veins of the valve-like arachnoid granulations in the sagittal sinuses. However, there is no direct evidence that there is lymphatic drainage of CSF except for one site (olfactory epithelium, to which CSF drains through the multiple olfactory nerves penetrating the highly perforated cribriform plate) (Walter B. A., Valera V. A., Takahashi S, and Ushiki T. Neuropathology and Applied Neurobiology (2006), 32,388-396). The “valve-like” structure of the arachnoid granulations suggests that CSF may move through these valves in one direction along the CSF-venous blood pressure gradient. However, while ex vivo (under artificially elevated pressure conditions) several direct connections between CSF and blood vessels have been observed (Johnston M, Armstrong D and Koh L. Cerebrospinal Fluid Research 2007, 4:3-8), there is no direct evidence that such connections exist at normal CSF and blood pressures in vivo. Mechanism notwithstanding, the data are in agreement with the significant direct transfer of proteins from CSF to the blood, bypassing the lymphatic system.

Since the mechanisms of CSF (water and solute) transfer to the blood are unknown, the published data on the small molecule and protein clearance from the leptomeningeal compartment are difficult to interpret and the behavior of molecules is very difficult to predict a priori. The data on protein clearance indicate a reverse correlation of the clearance rate with the molecular weight (size), the CSF half-life being from 30 min for small ones (Nagaraja T N, Patel P, Gorski M, Gorevic P D, Patlak C S and Fenstermacher J D. Cerebrospinal Fluid Research 2005, 2:16) to hours for the larger ones (Bergman I, Burckart G J, Pohl C R, Venkataramanan R, Barmada M A, Griffin J A And Cheung N-K V. WET 284:111-115, 1998; Betz A L, Goldstein G W and Katzman R Blood-brain-cerebrospinal fluid barriers, in Basic Neurochemistry: Molecular, Cellular, and Medical Aspects (Siegel G J, Agranoff B W, Albers R W, Molinoff P B. eds) pp 591-606, Raven Press, New York (1989)). On the other hand, some small molecules apparently have longer half-lives than proteins. For example, the indium complex of diethylenetriaminepentaacetic acid (In-DTPA, widely used in cysternography) has a 13.5±4.5 hours half-life in humans. Some compounds, such as In-DTPA, can reach brain ventricles in humans ca. 5 hours after intralumbar injection (human data) (Takahashi K and Mima T. Cerebrospinal Fluid Research 2009, 6:5), while other compounds can only reach the cerebral compartment if injected in a volume exceeding 10% of the total CSF volume (humans and monkeys) (Rieselbach R, DiChiro G, Freireich E J, and Rall D P. New Engl. J. Med. 1962, 267:1273-1278).

Other data suggest that proteins can be withdrawn from CSF not only through drainage with the liquid phase, but also if they can bind the surrounding epithelium. For example, horseradish peroxidase conjugated wheat germ agglutinin and administered into the CSF in mice was bound by the epithelial cell surface and transported to the capillary surface of the choroid plexus within 10 min after the injection (Balin B J and Broadwell R D (1988). Neurocytology 17:809-826).

Although these data are still too fragmentary to be systematically analyzed, the present disclosure encompasses the recognition that (1) large molecules can remain in the CSF for a long time (unless they can be cleared by the surrounding tissues); (2) small molecules, unless they are withdrawn by transporter/receptor specific mechanisms or hydrophobic enough to diffuse through the leptomeningeal tissues directly to the abundant capillary vessels, are cleared from CSF approximately with the rate of CSF turnover; (3) injection into cysterna magna or ventricles results in drug distribution in the entire CSF volume, and (4) injection into the lumbar fluid can result in drug translocation to the cerebral CSF compartment, even apparently against the CSF flux. The clearance of large molecules from the CSF also (5) appears to depend on their molecular weight (hydrodynamic size).

While not wishing to be bound by any particular theory, it is proposed that a drug that would remain in the CSF for a sufficiently long period of time (several hours or days) and have access to cancer cells would efficiently terminate or slow the meningeal cancer spread and greatly improve the outcome of chemotherapy. However, conventional drugs, being small molecules, rapidly leave CSF for systemic circulation.

Our recent studies on the behavior of large molecules in CSF clearly show that they are not nearly as rapidly cleared from CSF. Being administered intrathecally, they rapidly distribute over the entire CSF volume and stay there for several hours or days. This inspired us to develop soluble large-molecule therapeutics that would distribute in CSF and release an insoluble antineoplastic drug that would readily access the meningeal population of cancer cells and also stay in the meninges. In some embodiments, provided methods employ conjugates comprising a biocompatible carrier of a sufficiently large hydrodynamic size, the carrier releasing a water-insoluble chemotherapeutic drug (or prodrug) over a sufficiently long period of time such that administration of the conjugate to CSF efficiently suppresses and/or eradicates the presence and/or spread of leptomeningeal cancer. In some embodiments, a released form of a drug or prodrug is highly hydrophobic or otherwise capable of association with the leptomeningeal (or a nearby) compartment, thereby prolonging the residence and/or activity of the drug or prodrug in the target area.

Such intrathecal therapeutics would (i) distribute with CSF over the compartment where the cancer cells reside, then (ii) release the drug at the concentration that would kill cancer cells, and (iii) would not rapidly penetrate to the systemic circulation and thus would not cause any systemic side effects. It may be desirable to release a hydrophobic prodrug that would be activated over time: thus the action of the drug is prolonged and the time profile of the drug concentration is flattened, reducing the peak level (Cmax) in the CSF and thus reducing the local toxicity.

Camptothecin (as well as its synthetic analogs—tecans) is a Topoisomerase I inhibitor. The activity of tecans against several cancer cell lines is well documented. The drawbacks of the presently available tecans are rooted in their pharmacokinetics. The results of the Phase I studies of a camptothecin conjugate (Sausville et al. A Phase 1 study of XMT-1001, a novel water soluble camptothecin conjugate, given as an intraveneous infusion once every three weeks to patients with advanced solid tumors, Proceedings of AACR-NCI-EORTC International Conference Molecular Targets and Cancer Therapeutics, 2009 (November 2009) abstr B52) confirm that the toxicity of free CPT in humans, which prevents its use as a drug, has its foundation in the pharmacokinetics of CPT rather than in the molecular mechanism of the drug action. Once the pharmacokinetics is optimized (via the design of a conjugate of an optimal size equipped with an optimized drug release system), the characteristic toxicity of CPT (induction of hemorrhagic cystitis) is fully eliminated without the loss of therapeutic activity.

In some embodiments, provided methods comprise intrathecal administration of a CPT-based macromolecule in a sufficient amount to prevent or slow the meningeal spread of tecan-sensitive cancer.

We have developed one soluble large molecule conjugate that releases a highly hydrophobic prodrug form of camptothecin (CPT) (see U.S. Patent Application Publication No. 2007/0190018). This can be used as a prototype for the meningeal drugs described herein.

In some embodiments, the present invention provides conjugates for use in medicine. In certain embodiments, the present invention provides methods for treating cerebral, meningeal or neural diseases, conditions, and disorders. In some embodiments, the disease is a cancer. In some embodiments, the disease is an infectious disease. In some embodiments, the disease is caused by a genetic deficiency. In certain embodiments, the disease is a disease of the brain, spinal cord, large nerves, meninges, or a combination thereof. In some embodiments, the disease is a disease of tissues directly or indirectly contacting the cerebrospinal fluid. In certain embodiments, the present invention provides methods of treating pain.

In some embodiments, the disease, condition, or disorder is of the brain. Example of such brain diseases include, for example, genetic deficiencies, cancer, trauma, stroke, infections (viral and bacterial), and various geriatric conditions. Diseases of the spinal cord of the similar origins may also be treated. Pain of cerebral, meningeal, or neural tissues may also be treated by methods and conjugates of the present invention.

Diseases of the meninges treatable by methods of the present invention are also multiple and include cancer (neoplastic meningitis, meningiomas), infections (viral, bacterial, fungal), trauma, hemorrhages, and chemically induced conditions.

Diseases of the large nerves treatable by methods of the present invention include traumatic conditions, autoimmune damage, infection (neuritis), diabetic damage; and damage caused by vitamin B12 deficiency.

The above lists are not complete and intended only for illustration of diseases and conditions for which the present invention can provide new treatments. Currently, there are no safe, effective treatments for many of such conditions.

The brain, spinal cord and large nerves are well isolated from the rest of the body by a known system of barriers (e.g., blood-brain barrier, blood-CSF barrier, meninges, endoneurum, myelin sheaths). Several attempts have been made to improve drug delivery through the barriers to the site of disease, with various degree of success.

In certain embodiments, conjugates are used in methods of treating animals (preferably mammals, most preferably humans). In some embodiments, conjugates of the present invention may be used in a method of treating animals which comprises administering to the animal a biodegradable biocompatible conjugate of the invention. For example, conjugates in accordance with the invention can be administered in the form of soluble linear polymers, copolymers, conjugates, colloids, particles, gels, solid items, fibers, films, etc. Biodegradable biocompatible conjugates of this invention can be used as drug carriers and drug carrier components, in systems of controlled drug release, preparations for low-invasive surgical procedures, etc. Pharmaceutical formulations can be injectable, implantable, etc.

In some embodiments, the invention provides methods of treating a disease or disorder in a subject in need thereof, comprising administering to the subject an efficient amount of at least one conjugate of the invention; wherein said conjugate releases one or more modifiers in a dual phase process; wherein said modifier(s) is(are) suitable therapeutic agent(s) for the treatment of the disease or disorder.

In some embodiments, the invention relates to the treatment of a “neurological disease, disorder, or damage,” which is a disease, damage, or disorder that results in the disturbance in the structure or function of the central or peripheral nervous system resulting from developmental abnormality, disease, injury or toxin. Examples of neurological diseases or disorders include, but are not limited to, neurodegenerative disorders (e.g. associated with Parkinson's disease or Parkinsonian disorders, Alzheimer's disease, Huntington's disease, Shy-Drager Syndrome, Progressive Supranuclear Palsy, Lewy Body Disease or Amyotrophic Lateral Sclerosis); ischemic disorders (e.g. cerebral or spinal cord infarction and ischemia, stroke); traumas (e.g. caused by physical injury or surgery, and compression injuries; affective disorders (e.g. stress, depression and post-traumatic depression); neuropsychiatric disorders (e.g. schizophrenia or other psychoses, multiple sclerosis or epilepsy); radiation damage, lysosomal storage diseases, and learning and memory disorders.

In some embodiments, a disease or disorder of the central nervous system is selected from the group consisting of cancer-related brain/spinal cord injury or diseases or disorders of the nervous system, including, but not limited to, lissencephaly syndrome, depression, bipolar depression/disorder, anxiety syndromes/disorders, phobias, stress and related syndromes, cognitive function disorders, aggression, drug and alcohol abuse, obsessive compulsive behavior syndromes, seasonal mood disorder, borderline personality disorder, cerebral palsy, drug addictions, multi-infarct dementia, Lewy body dementia, age related/geriatric dementia, epilepsy and injury related to epilepsy, temporal lobe epilepsy, brain injury, trauma related brain/spinal cord injury, anti-cancer treatment related brain/spinal cord tissue injury, infection and inflammation related brain/spinal cord injury, environmental toxin related brain/spinal cord injury, autism, attention deficit disorders, narcolepsy, and sleep disorders.

In some embodiments, the reference to disease, damage, or disorder of the nervous system is selected from the group consisting of neurodegenerative disorders, neural stem cell disorders, neural progenitor disorders, ischemic disorders, neurological traumas, affective disorders, neuropsychiatric disorders, degenerative diseases of the retina, retinal injury/trauma and learning and memory disorders.

In certain embodiments, a disorder is related to a mineral or vitamin deficiency. In some embodiments, a disorder is pernicious anemia.

Despite the significant recent improvements in cancer statistics in the US, cancer remains one of the major causes of death. The efficacy of chemotherapy, which is the major therapeutic modality, is still limited by the toxicity of the available drugs that hinders dose elevation to the levels resulting in reliable remission. One aspect of the present invention relates to the possibility of developing new, considerably more efficient and less toxic chemotherapeutic preparations. The inventive system can also be useful in inflammation, pain management, and, generally, in all other areas where various sustained release or targeting of drugs is beneficial.

Macromolecular drug delivery systems, which have been extensively studied over the past two decades, significantly improved the pharmacological properties of several drug substances, and provided new tools for controlling drug delivery to cancer cells. A vast majority of the antineoplastic drug conjugates reported so far (a) are inactive until the drug substance is released from the macromolecular carrier, and (b) the drug substance is released, or at least intended to be released, in one stage. In some cases, the conjugate (e.g., of a protein) may be active without drug release from the carrier.

Benefits of drug association with carrier macromolecules relate, in part, to the following factors: (1) solubilization of the drug substance; (2) restricted drug substance access to normal interstitium due to the large hydrodynamic size of the conjugate, (3) conjugate delivery to the tumor tissues via the Enhanced Permeability and Retention (EPR) effect, and (4) maintenance of sustained drug levels over periods exceeding cancer cell cycle. In some conjugates, the specificity of drug delivery to cancer cells is further addressed via incorporation of various targeting moieties (e.g., antibodies), and via enzyme-assisted hydrolysis of the link connecting the drug molecule to the carrier. The above benefits generally relate to the systemic administration of conjugates. In the present invention, conjugation is useful in that it provides better exposure of the CSF and the surrounding tissues to the drug due to the distribution of the conjugate over the CSF compartment and prolonged residence therein. Conjugation is further useful by providing benefits of drug solubilization, enhanced penetration into the damaged tissues from CSF, and maintenance of sustained drug concentration over a prolonged period of time.

In several preclinical studies, antineoplastic drug conjugates were shown to be less toxic than respective free drugs. Antineoplastic activity of the conjugates (per unit of the administered drug substance) was usually lower than of unmodified drugs, although in some cases similar or higher. However, conjugates are frequently more effective at equitoxic doses, so the partial loss of antineoplastic activity is outweighed by the lower toxicity and larger maximal tolerated doses.

In certain embodiments, any or more of the methods described above further comprises administering at least one additional biologically active compound.

In certain embodiments, a modifier and biologically active compound are independently selected from the group consisting of vitamins, anti-AIDS substances, anti-cancer substances, radionuclides, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, radioprotectors, antioxidants, antidotes, growth factors, enzymes, cytokines, imaging agents, and combinations thereof.

In certain embodiments, in practicing methods of the invention, a conjugate further comprises or is associated with a diagnostic label. In certain embodiments, the diagnostic label is selected from the group consisting of: radiopharmaceutical or radioactive isotopes for gamma scintigraphy and PET, contrast agent for Magnetic Resonance Imaging (MRI), contrast agent for computed tomography, contrast agent for X-ray imaging method, agent for ultrasound diagnostic method, agent for neutron activation, moiety which can reflect, scatter or affect X-rays, ultrasounds, radiowaves and microwaves and fluorophores. In certain embodiments, the conjugate is further monitored in vivo.

In some embodiments, the invention provides a method of treating a disease or disorder in a subject, comprising preparing an aqueous formulation of at least one conjugate of the invention and parenterally injecting said formulation in the subject. In certain exemplary embodiments, a conjugate comprises a biologically active modifier. In certain exemplary embodiments, a conjugate comprises a detectable modifier.

In some embodiments, the invention provides methods of treating a disease or disorder in a subject, comprising preparing an implant comprising at least one conjugate of the invention, and implanting said implant into the subject. In certain exemplary embodiments, the implant is a biodegradable gel matrix.

In some embodiments, the present invention provides methods of providing therapy or neuroprotection to a patient in need thereof for treating Alzheimer's disease, Parkinson's disease, amyotrophic lateral sclerosis, multiple sclerosis, HIV neuropathy, Guillain-Barre' syndrome, neural transplantation, neural xenotransplantation, stroke, brain hemorrhage, brain and spine trauma, ionizing radiation, neurotoxicity of vestibular structures, or retinal detachment, which comprises administering to said patient a conjugate as described above; wherein the conjugate is administered directly into the cerebrospinal fluid space of a patient; wherein the linker is characterized in that it releases the modifier into the cerebrospinal fluid space at a rate sufficient to provide an efficient amount of the modifier; and wherein the conjugate displays continued residence in the cerebrospinal fluid for at least 30 minutes.

In some embodiments, the invention provides methods for treating of an animal in need thereof, comprising administering a conjugate according to the methods described above, wherein said conjugate comprises a biologically active modifier. In certain exemplary embodiments, the biologically active component is a gene vector.

In certain embodiments, a conjugate is associated with a diagnostic label for in vivo monitoring.

A physician or veterinarian having ordinary skill in the art can readily determine and prescribe the effective amount of the conjugate required. For example, the physician or veterinarian could start doses of the modifiers of the invention employed in the conjugate at levels lower than that required to achieve the desired therapeutic effect and then gradually increasing the dosage until the desired effect is achieved.

In some embodiments, a conjugate of the invention is provided to a subject chronically. Chronic treatments include any form of repeated administration for an extended period of time, such as repeated administrations for one or more months, between a month and a year, one or more years, or longer. In many embodiments, a chronic treatment involves administering a conjugate of the invention repeatedly over the life of the subject. Preferred chronic treatments involve regular administrations, for example one or more times a day, one or more times a week, or one or more times a month. In some embodiments, a suitable dose such as a daily dose of a conjugate of the invention will be that amount of a released modifier that is the lowest dose effective to produce a therapeutic effect. Such an effective dose will generally depend upon the factors described above. Generally doses of a modifier of this invention for a patient, when used for the indicated effects, will range from about 0.0001 to about 100 mg per kg of body weight per day. Preferably the daily dosage will range from 0.001 to 50 mg of modifier per kg of body weight, and even more preferably from 0.01 to 10 mg of modifier per kg of body weight. However, lower or higher doses can be used. In some embodiments, the dose administered to a subject may be modified as the physiology of the subject changes due to age, disease progression, weight, or other factors.

The therapeutically effective amount of a modifer will vary depending on the patient, the disease being treated, extent of disease, the rate of release from a carrier, other medications being administered to the patient, desired outcome, etc. It will be appreciated that the administered dose of a modifier can be calculated based on the ratio (mass, volume, molar, etc.) of a modifier to a conjugate (and optionally a linker). In certain embodiments, a modifer is administered in the range of approximately 0.00001 mg/kg body weight to approximately 10 mg/kg body weight. In certain embodiments, a modifer is administered in the range of approximately 0.0001 mg/kg body weight to approximately 1 mg/kg body weight. In certain embodiments, a modifer is administered in the range of approximately 0.001 mg/kg body weight to approximately 1 mg/kg body weight. In certain embodiments, a modifer is administered in the range of approximately 0.0001 mg/kg body weight to approximately 0.001 mg/kg body weight. In certain embodiments, a modifer is administered in the range of approximately 0.0001 mg/kg body weight to approximately 0.01 mg/kg body weight. In certain embodiments, a modifer is administered in the range of approximately 0.0001 mg/kg body weight to approximately 0.1 mg/kg body weight. In certain embodiments, a modifer is administered in the range of approximately 0.001 mg/kg body weight to approximately 0.1 mg/kg body weight. In certain embodiments, a modifer is administered in the range of approximately 0.01 mg/kg body weight to approximately 0.1 mg/kg body weight. However, lower or higher doses may be used. Such doses may correspond to doses found useful and appropriate in an applicable animal model (e.g., in a transgenic rodent model). Such dosages useful in an experimental model may range from about 1 mg/kg to about 0.001 mg/kg. In certain embodiments, the dosage in an experimental animal ranges from about 1 mg/kg to about 0.01 mg/kg. In certain embodiments, the dosage in an experimental animal ranges from about 0.5 mg/kg to about 0.01 mg/kg. In certain embodiments, the dosage used in an applicable animal model is approximately 0.01 mg/kg, approximately 0.02 mg/kg, approximately 0.03 mg/kg, approximately 0.04 mg/kg, approximately 0.05 mg/kg, approximately 0.06 mg/kg, approximately 0.07 mg/kg, approximately 0.08 mg/kg, approximately 0.09 mg/kg, or approximately 0.1 mg/kg. In some embodiments, the dose administered to a subject may be modified as the physiology of the subject changes due to age, disease progression, weight, or other factors.

If desired, the effective daily dose of an active modifier may be administered as two, three, four, five, six, or more sub-doses administered separately at appropriate intervals throughout the day, optionally, in unit dosage forms.

A conjugate according to the invention may be formulated for administration in any convenient way for use in human or veterinary medicine, by analogy with other pharmaceuticals.

Examples of applications to drug delivery methods applicable to the present invention can be found inter alia in International Patent Application Publications WO 2003/59988, WO 2005/023294, WO 2004/009082, WO 1996/032419, WO 2001/010468, and U.S. Pat. No. 7,160,924, the entirety of each of which is hereby incorporated by reference. These include polyal-small-molecule-drug conjugates, protein-modified carriers, cationized polyal, polyal-modified liposomes, polyal-modified nano- and microparticles.

In some embodiments, the biodegradable biocompatible conjugates of the present invention can be monitored in vivo by suitable diagnostic procedures. Such diagnostic procedures include nuclear magnetic resonance imaging (NMR), magnetic resonance imaging (MRI), ultrasound, X-ray, scintigraphy, positron emission tomography (PET), etc. The diagnostic procedure can detect, for example, conjugate disposition (e.g., distribution, localization, density, etc.) or the release of drugs, prodrugs, biologically active compounds or diagnostic labels from the biodegradable biocompatible conjugate over a period of time. Suitability of the method largely depends on the form of the administered conjugate and the presence of detectable labels. For example, the size and shape of conjugate implants can be determined non-invasively by NMR imaging, ultrasound tomography, or X-ray (“computed”) tomography. Distribution of soluble conjugate preparation comprising a gamma emitting or positron emitting radiotracer can be performed using gamma scintigraphy or PET, respectively. Microdistribution of conjugate preparation comprising a fluorescent or scattering label can be investigated using photoimaging.

It is understood, for the purpose of this invention, that transfer and disposition of conjugates in vivo can be regulated by modifying groups incorporated into the conjugate structure, such as hydrophobic and hydrophilic modifiers, charge modifiers, receptor ligands, antibodies, etc. Such modification, in combination with incorporation of diagnostic labels, can be used for development of new useful diagnostic agents. The latter can be designed on a rational basis (e.g., conjugates of large or small molecules binding known tissue components, such as cell receptors, surface antigens, etc.), as well as through screening of libraries of conjugate molecules modified with a variety of moieties with unknown or poorly known binding activities, such as synthetic peptides and oligonucleotides, small organic and metalloorganic molecules, etc.

It will be appreciated that for the foregoing description of methods utilizing conjugates, the present invention encompasses the composition of such conjugates.

### EXEMPLIFICATION

### General Procedures

Chemical data is processed to determine yields, size distributions, composition and (for solutions) substance content. Data is tabulated and, where applicable, statistically processed to determine mean values and standard deviations. Spectral data (NMR, UV/VIS) is obtained and analyzed to confirm chemical composition of the synthesized preparations. Size exclusion HPLC is used to determine the molecular weight/size distributions. All size exclusion columns are double-calibrated with (a) protein standards with known hydrodynamic diameters, and/or (b) linear polymer (PEG) standards with known molecular weights. Since biological selectivity is dependent on the hypothetical size-selective sieving mechanism, it is expected that in vivo behavior will better correlate with the hydrodynamic sizes of carrier coils than with the molecular weights. The size exclusion HPLC profiles of all obtained materials may be recalculated into hydrodynamic size distributions and also expressed in terms of M_{w}, Mₙ and polydispersity index (PDI).

Radiochemical data are processed analogously. Radiochemical purity is assessed based on HPLC data (gamma detection). The radioactivity of all solutions intended for animal studies is measured on a well counter that is calibrated using a standard with a known (measured on a photon counting detector) ¹²⁴I dose. All radioactivity data is corrected for iodine decay.

Imaging: The imager (MicroPet P4, Concord Microsystems) is calibrated with a volume phantom containing a known concentration of ¹²⁴I (measured on a calibrated well counter). Images are reconstructed using OSEM3D/MAP protocol. Regions of interest are drawn manually. The numerical data from the ROIs are statistically processed to determine mean values and standard deviations for agent accumulation in each tissue or organ in all groups of animals used in the experiment. The data are further processed to determine the kinetic parameters of the material translocation and the degree of data correlation with the physiological mathematical model.

Toxicity and efficacy studies: Averages and standard deviations may be calculated where applicable.

### Example 1

### Behavior of Albumin and RNAse after Intrathecal Administration

Model macromolecules, bovine serum albumin and RNAse, were labeled with I-124 and injected into anesthetized male and female SD rats into cisterna magna through the atlanto-occipital membrane. Rats were immediately placed on the imaging bed of MicroPET P4 imager.

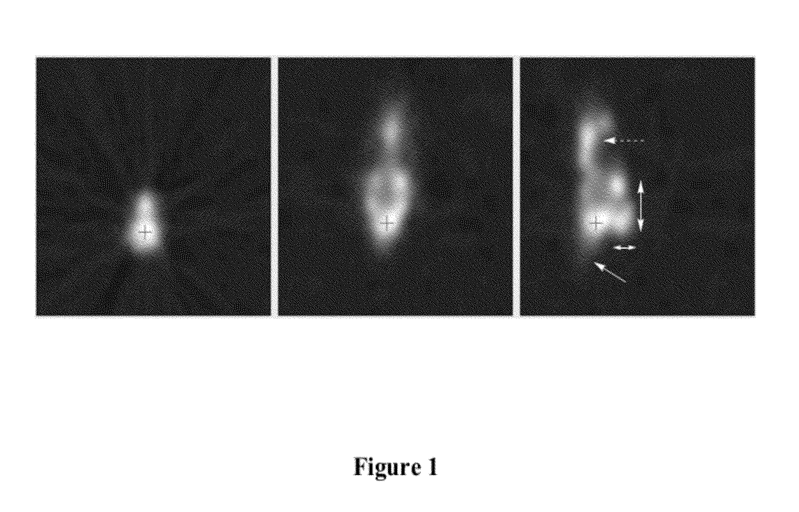

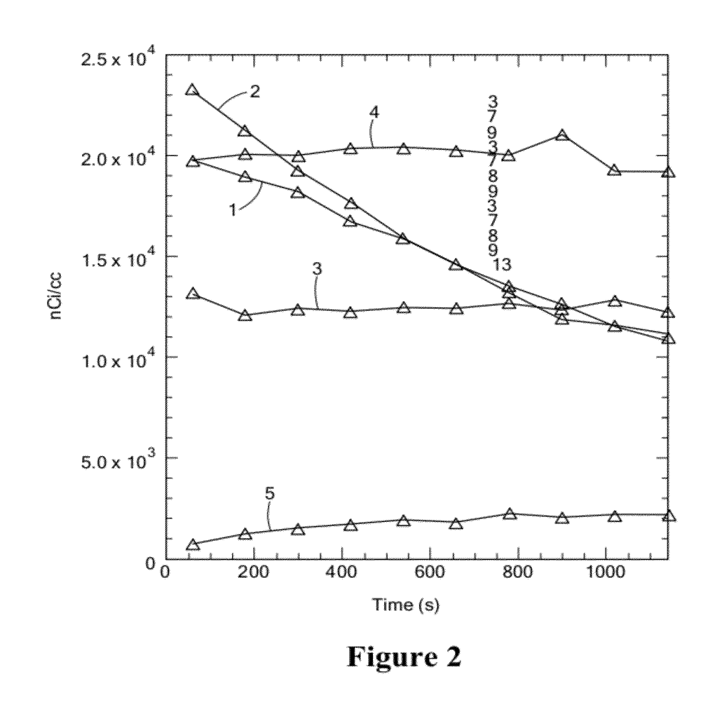

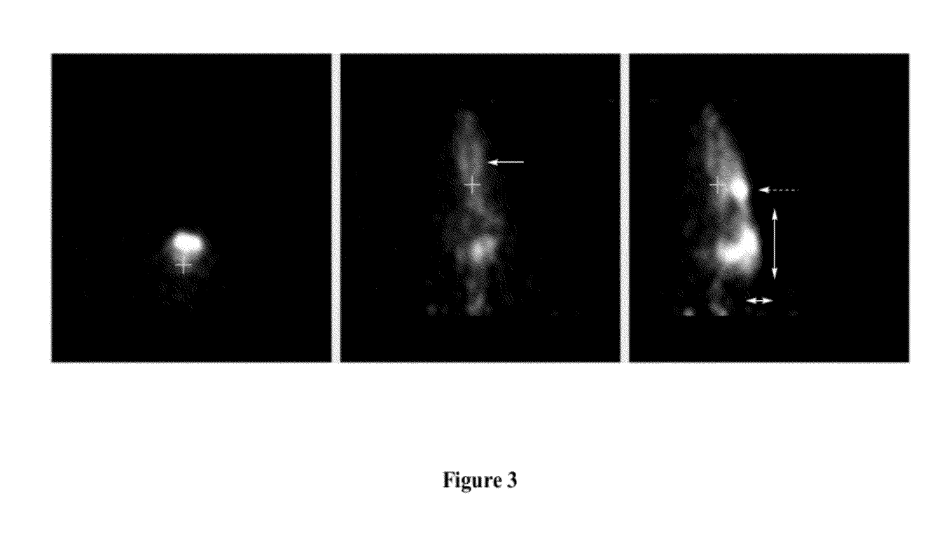

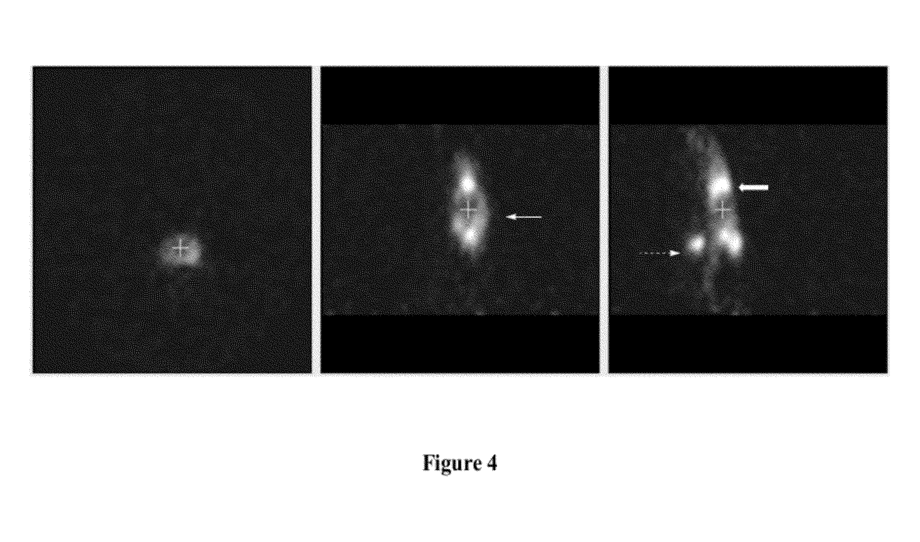

Imaging data acquisition started immediately after the injection and continued for 20 min. The data were reconstructed as dynamic sequences, 2 minutes per frame. Then, rats were reimaged at 2, 4, 8, and 24 hours. Image analysis demonstrated that:
the injected solution rapidly distributes in CSF, including areas well beyond brain and spinal cord (presumably along major nerves), see FIGS. 1 and 2.
The model molecules stay in the CSF compartment, with very slow transfer to the outside that apparently depends on the molecular weight, see FIGS. 3 and 4.

### Example 2

This Example sets forth the procedure used to determine the size of the drug molecule that would enable its optimal retention in CSF, synthesize a model conjugate, and evaluate in animal models drug distribution in the meningeal compartment, efficacy against meningeal cancer spread, and safety.

### Procedure

In order to determine the optimal molecular (hydrodynamic) size of the drug conjugate and synthesize a model chemotherapeutic conjugate of that size, the following steps are taken:
1.1. Synthesize carrier molecules of PHF of varying size, from 3 to 15 nm, labeled with ¹²⁴I.
1.2. Investigate the dependence of the retention of radiolabeled molecules of 1.1 in CSF on the molecule size (rats and monkeys, PET imaging). Select the optimal carrier molecule size.
1.3. Synthesize a model conjugate of PHF and camptothecin of the optimal size, as determined in 1.2.

In order to evaluate the efficacy and safety of the model conjugate in an animal model of meningeal cancer spread, the following steps are taken:
2.1. Determine the MTD of the synthesized conjugate in mice.
2.2. Label the drug molecule incorporated into the conjugate (camptothecin) with ¹²⁴I and investigate the dynamics of drug distribution and deposition over the CSF compartment by PET.
2.3. Investigate the efficacy of the conjugate in a meningeal cancer spread model.

The strategy described herein establishes whether a carrier molecule of a sufficiently large size releasing a water-insoluble chemotherapeutic drug (or prodrug) over a sufficiently long period of time will, being injected to CSF, efficiently suppress and/or eradicate the meningeal cancer spread.

Accordingly, it is first determined how large the carrier molecule should be in order to be confined to the CSF compartment. Next, a model molecule of that size carrying a model drug is prepared, and its safety and efficacy is evaluated in an animal model. This strategy extensively utilizes PET imaging, which is the best available quantitative tool for real-time tracking of drug molecule transfer in vivo (which is useful for this strategy). All data, where appropriate, is analyzed statistically (mean values, standard deviations, p values).

### Molecule Size and Retention in the CSF.

Prior to the present disclosure, the mechanism of retention of the large molecules in CSF was not known. Elsewhere in the body, several barriers are known to be size selective. Their molecular mechanisms include either true pores (small vascular pores, merged endosomal or caveolar “pipes”), or active processes acting as pores (transcytosis). Even for the best known barriers, such as the renal glomerular “filter”, the exact mechanisms of filtration are not yet known, and these filters may not be simple “molecular sieves”. For example, recently a permeation/diffusion mechanism was suggested for the glomerular filter that explains why the “sieve” never clogs (Smithies O. PNAS 2003, 100:4108-13). Regardless of the mechanism, all size-selective barriers (in vivo as well as in vitro) have S-shaped permeability profiles: the permeability changes within a relatively narrow “cut-off” range of molecular sizes (weights) that corresponds to the effective size of the “pores”. Molecules of a smaller size penetrate through the pores freely, while molecules of a significantly larger size don't penetrate at all. Filters with narrow pore size distribution generally have a more narrow cutoff range that ones with a broad pore size distribution (Jaeckle K A. Semin Oncol. 2006, 33:312-23).

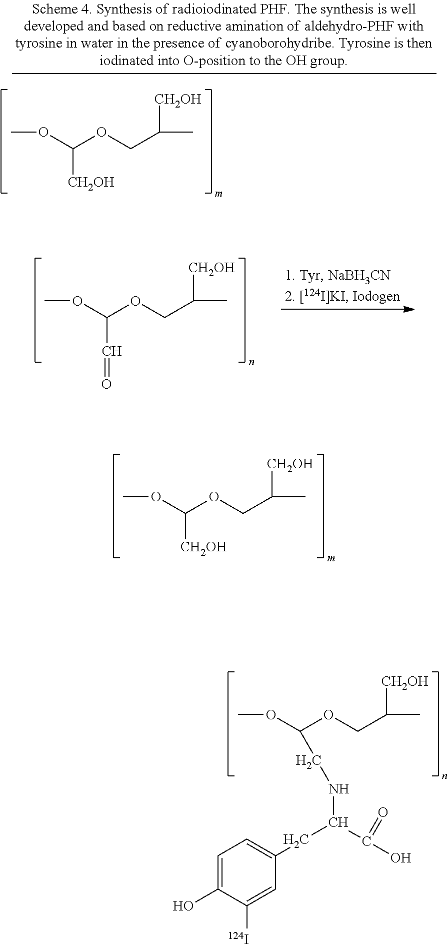

One step of this strategy is to determine the high molecular weight/size boundary of the cut-off range of the “filter” that retains large molecules in the CSF. Going beyond this range would not result in a significant gain in drug retention in CSF, but would result in a higher viscosity that may impair CSF circulation. Based on published preliminary data obtained in rats with five model proteins of different sizes (RNAse, Idursulfase, ARSA, HNS, bovine albumine), the retention of macromolecules in CSF is increasing in the range of 2-10 nm, and the maximal retention can be expected for molecules larger than 10 nm in hydrodynamic diameter. It is expected that highly fractionated PHF, which is a non-toxic, biodegradable polymer suitable for human use, will behave in a manner similar to that observed with these model proteins. PHF, a semi-synthetic copolymer of glycerol and glycol aldehyde, (Papisov M, Yurkovetskiy A, Hiller A, Yin M, Barzana M, Hillier S, and Fischman A J. Biomacromolecules 2005, 6:2659-2670) has been successfully tested in several other animal models and, most recently, in Phase I clinical trials (Sausville E A, Anthony S P, Garbo L E, Shkolny D, Yurkovetskiy A V, Bethune C, Schwertschlag U, Fram R J. Abstract A146, AACR-NCI-EORTC International Conference on Molecular Targets and Therapeutics, San Francisco, Calif., 2007; Drug Data Report 2007, v. 29(5), p. 445), with no observable side effects. In this experiment, PHF is conjugated with a small amount of tyrosine (1 tyrosine moiety per 20 monomer units) and label the tyrosine with ¹²⁴I using Iodogen as an iodination reagent (Scheme 4, m:n=20:1).

### Synthesis

Using previously developed methods, six samples of highly fractionated PHF glycol are prepared. Fractionation is carried out on a multigram scale by flow dialysis. Then, the crude fractions are subfractionated by size exclusion HPLC on Sephacryl S-100 and lyophilized. The resultant PHF glycol is converted to aldehydro-PHF by brief treatment with periodate under mild conditions (25° C., pH=7, 10 min), which occurs with preservation of the molecular size (no depolymerization or crosslinking). The product, aldehydro-PHF, is desalted on Sephadex 25 and, if necessary, concentrated by flow dialysis or ultrafiltration on a 3 kDa membrane. The aldehydro-PHF is then conjugated with tyrosine by reductive amination at pH=8, which is not expected to result in a significant alteration in the molecular size. The product is analyzed for the latter by size exclusion HPLC and, if necessary, re-fractionated on a preparative size exclusion HPLC column. The product is concentrated on a membrane filter with a 3 kDa cutoff and stored at 4° C. A fraction of the product is lyophilized and analyzed for the incorporation of tyrosine by UV spectrometry (the expected incorporation is between 20 and 40 PHF monomer units per tyrosine residue). All work with tyrosine is carried out under argon to avoid oxidation of the phenol OH group.

Tyrosine-PHF is radioiodinated immediately before the injection. The iodination is carried out at pH=7 (0.1 M phosphate buffer solution), 25° C., ¹²⁴I using Iodogen as an iodination reagent. The polymer does not contain any oxidation-sensitive structures; therefore, no depolymerization occurs under these conditions. The product is evaluated by size exclusion HPLC with double (gamma and UV) detection, and, if necessary, subfractionated on a semi-preparative HPLC column. Alternatively and/or additionally, the product is desalted on Sephadex 25, concentrated on a 3 kDa membrane in saline, and used in animal studies within 1 hour. The amount of substance in the solution is determined from the HPLC UV data (by tyrosine absorption at 280 nm). The radioactivity is determined using a calibrated well counter (dose calibrator).

The synthesis has been described in our previous publications (see Scheme 5, below). For example, camptothecin is conjugated with BOC-protected glycinate, then deprotected to obtain CPT-glycinate. PHF is treated with succinic anhydride to obtain PHF succinate with ca. 10% monomer units succinylated. The latter is conjugated with CPT-glycinate to obtain the final product, PHF-CPT (see Scheme 5, below). The product is characterized by size exclusion HPLC and proton NMR (the molecular weight/size distribution, content of PHF, glycine, succinate and CPT. The product is stable at 4<pH<6.5. In the event the molecular size distribution deteriorates and/or is found to be suboptimal, the product may be refractionated under these conditions.

The product is lyophilized, stored at −80° C., and reconstituted for animal studies in 10 mM citrate buffered saline, pH=6.8, immediately before the injection. Intrathecal administration of slightly acidic solutions with low buffer capacity has not been observed to cause toxicity or any observable side effects.

The molecular sizes and the radiochemical purities of the radioiodinated PHF are investigated by size exclusion HPLC.

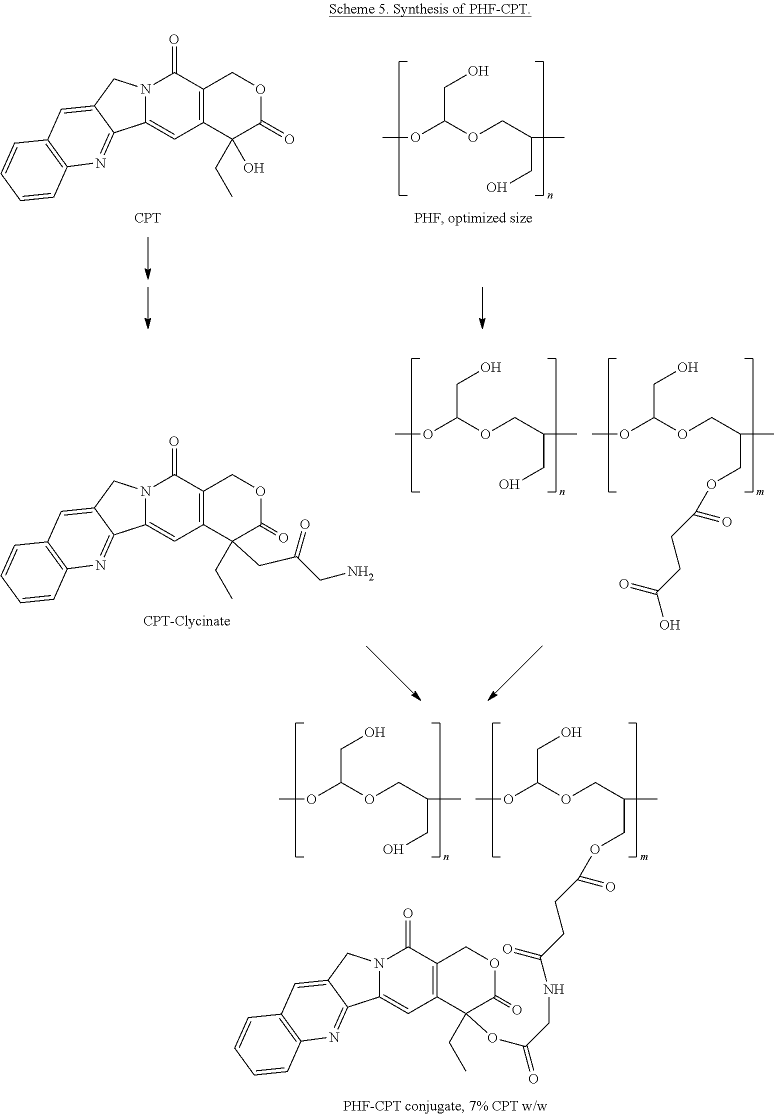

### Example 3

### Investigation of large molecule translocations in CSF by PET with ¹²⁴I

Applicant's studies described in this Example are also described by Belov et al. and Papisov et al, Abstracts of the annual meeting of the Society of Nuclear Medicine, Toronto, 2009. With the growing number of biotechnology products entering preclinical and clinical studies, PET imaging of slow pharmacokinetics (PK) is playing an increasingly important role. Among all currently available positron emitters suitable for long-term (several days) PET studies, ¹²⁴I has the longest physical half-life (4.2 d). The objective of this Example is to exemplify the properties of ¹²⁴I as a “non-pure” positron emitter translate into data quality suitable for PK studies.

Imaging was performed using MicroPET P4. Spatial resolution (full width at half maximum, FWHM) was studied using a line ¹²⁴I source (Ø=0.19 mm) in water. A 51×127 mm cylindrical phantom was used to evaluate the count-rate performance and coincidence detection. Studies in rats and cynomolgus monkeys were carried out using five human recombinant enzymes. The proteins were labeled with ¹²⁴I, at up to 5 mCi/mg.

The transaxial and axial limiting spatial resolutions were satisfactory and higher with OSEM3D/MAP reconstruction than with filtered back projection (FBP), 2.4 vs. 3.3 mm, and 3.2 vs. 3.6 mm, respectively. A good linearity of the “true” coincidence count-rate, which is necessary for quantitative studies, was observed for activities of up to 1.2 mCi in the field of view. Animal studies demonstrated excellent delineation and resolution of even small organs (e.g., single lymph nodes in rats, Ø<1 mm) The quality of numerical data was appropriate for PK analysis over at least 8 days.

Thus, it can be concluded that ¹²⁴I is an excellent label for quantitative investigation of large molecules (and molecules with slow PK in general) by PET in rats and larger animals, but perhaps not always suitable for mice studies (V. Belov, A. A. Bonab, A. J. Fischman, M. Heartlein, P. Calias, M. Papisov. Iodine-124 as a Label for pharmacological PET imaging. Annual meeting of SNM, Toronto, CA, June 2009; V. Belov, A. A. Bonab, A. J. Fischman, M. Papisov. Iodine-124 as a PET Imaging Label for Pharmacokinetic Studies. 36th Annual Meeting of the Controlled Release Society, Copenhagen, Denmark, July 2009; M. Papisov, V. Belov, A. J. Fischman, A. A. Bonab, J. Titus, M. Wiles, H. Xie, M. Heartlein, P. Calias. PET Imaging of α-Galactosidase A Pharmacokinetics in Rats and Monkeys. Annual meeting of SNM, Toronto, CA, June 2009).

Next, three human recombinant enzymes, idursulfase, arylsulfatase A, and sulfamidase were labeled with ¹²⁴I and administered at 1 and 10 mg/kg via two routes, IV and IT. Dynamic imaging data and multiple static images were acquired over 8 days, and processed to determine the principal PK parameters. FITC labeled sulfamidase was also utilized to investigate brain cryosections by photoimaging.

The IT administration resulted in rapid protein distribution over the entire CSF volume, including distal spine. The initial label content in the brain region was 0.20%, 0.15% and 0.05% of the injected dose/g after IV and 45%, 70% and 35% after IT administration for idursulfase, arylsulfatase A, and sulfamidase, respectively.

Idursulfase was cleared from both the brain and spinal cord with a half-life of ca. 7 h, while the other two enzymes of ca. 24 h. Photoimaging studies indicated enzyme deposition in pia mater as well as in the deeper layers (M. Papisov, V. Belov, A. J. Fischman, A. A. Bonab, M. Wiles, H. Xie, M. Heartlein, P. Calias. PET Imaging of Enzyme pharmacokinetics in rats after IV and IT administration. Annual meeting of SNM, Toronto, CA, June 2009).

These data strongly suggest that a large molecule carrying an antineoplastic drug efficiently delivers the latter to the leptomeningeal compartment involved in the neoplastic meningitis, at least after CM (or intraventricular) administration. Both types of administration are clinically suitable, although not utilized as frequently as IL injection. The issue of the apparent macromolecule translocation “against the flow” after the IL administration is relevant to the feasibility of IL administration for drug delivery to the cerebral meninges.

### Example 4

### Animal Studies

### Rat Studies

Rats are anesthetized with sodium pentobarbital, 35 mg/kg IP. The animals are placed on an injection holder. The holder supports the animal body in a prone position with head tilted down at ca. 110° to the spine, which opens the atlanto-occipital joint for direct injection.

A polypropylene catheter equipped with a 30G needle is inserted through the atlanto-occipital membrane until CSF appears in the tubing. Then, the radioiodinated tyrosine-PHF is injected into cisterna magna through the catheter (n=6 animals per molecular weight of ¹²⁴I-PHF). The catheter is flushed with saline (20 μl). The dose is 1 mg/kg by the polymer, and 0.05-0.1 mCi by ¹²⁴I. Injection volume: 50 μl.

The rats are immediately placed on the MicroPET P4 imager bed in a prone position, and the injection site (head and upper body area, axial length of the field of view 64 mm) is imaged for 30 minutes, with subsequent dynamic image reconstruction. Then, static 5 minute whole body images are acquired at 2, 4, 8, 24, and 48 hours after the injection. The imaging schedule may be adjusted based on the early imaging data, as necessary.

The images are reconstructed using OSEM3D/MAP reconstruction protocol. This reconstruction requires a longer computation time but provides a 50% better resolution with ¹²⁴I than the more conventional filtered back projection (FORE-2DFBP).

The images are processed using Siemens ASIPro imaging software. In each image, PHF concentration is measured in the following regions of interest (ROI) of the images: whole head, cisterna magna, spinal column, heart, thyroid, stomach, liver, and kidneys. Other organs and tissues are measured if desired. This method provides data in essentially the same format as conventional biodistribution studies (i.e., PHF concentration in the ROI, PHF amount per organ or region).

The data are used to determine the kinetics of PHF macromolecule transfer (a) within the leptomeningeal space, and (b) from CSF to the systemic circulation. PHF retention in the meninges is plotted as graphs (% of retention vs. time) and described in terms of half-retention time or CSF half-life (time at which 50% of the injected dose is still retained in the meninges). The dependence of half-retention time on the molecular size of the PHF molecules is plotted and analyzed. The upper boundary of the retention cutoff is identified from the graph.

### Example 5

Non-human primate studies: CM administration: The radiolabeled PHF of the size that retains well in rats (upon the results of Example 4) is evaluated in cynomolgus monkeys (n=4, CM injection, two males and two females, 3 to 6 kg). This is a non-terminal experiment that will enable the following: (a) confirmation of the retention of PHF in the CSF in an animal biologically close to humans, and (b) evaluation of the dynamics of the macromolecule mixing with CSF and transfer over the leptomeningeal space in more detail than rats (due to the larger animal size).

The animals are sedated with Ketamine 20 mg/kg-Xylasine 2 mg/kg, intubated, and placed on a continuous intratracheal non-rebreathing Isoflurane/O₂ anesthesia system. The level of anesthesia is such that the animals are breathing without mechanical assistance. Heart and respiration rates and CO₂ content in the exhaled air are monitored continuously, and the flow of Isoflurane is adjusted if necessary.

The injection site is shaved, rinsed with 70% alcohol, and treated with betadine. A catheter equipped with a 25 gauge 1″ needle and a sealed injection cap is used. The needle is inserted until CSF flows into the catheter. A volume of CSF equal to the injection volume is drawn; then the agent is injected into the catheter through the cap, and the catheter is flushed with the withdrawn CSF and removed. The total administered dose does exceed 3 mg of PHF and 1 mCi of ¹²⁴I in a 50 μl volume in 0.9% sterile saline.

Imaging is carried out as described for rats, or alternatively the animal is immediately placed on a heated imaging bed in supine position, and the injection site is imaged for 5 minutes. Then, images of the adjacent body sections (along the aneroposterior axis) are imaged for 5 minutes each. The animal is repeatedly imaged, section by section (64 mm each) in the anteroposterior direction for 1 hour, to follow the translocation of the injected substance in CSF. Whole body images are then acquired at 4, 8, 24 and 48 hours (and at later time points if necessary) to investigate PHF distribution inside and outside of the leptomeningeal compartment. Transmission images are acquired at each imaging session for the PET image correction for attenuation.

All images are reconstructed using OSEM3D/MAP reconstruction protocol with attenuation correction. The images are processed as described in Example 4 to obtain quantitative data on PHF translocation within CSF and to the outside of the leptomeningeal compartment. The final data include PHF concentration (as a function of time) in cisterna magna, ventricles, spinal fluid, and other sub-compartments.

### Example 6

### Safety and Efficacy of PHF-CPT Conjugate in Animal Model of Neoplastic Meningitis

This Example describes how to develop the initial data on the orders of efficacy and toxicity of a model hydrophilic conjugate releasing a highly hydrophobic drug in the leptomeningeal space. While there is no reason to believe PHF-CPT is the only possible candidate for the neoplastic meningitis targeted drug, it is used here as a model candidate because it is a system that is already developed and well characterized by the Applicant, comprising components with known safety profiles, including Phase I human data. Other polymers, release systems, and drug substances may be similarly tested.

Experimental drug substance: camptothecin. Camptothecin (as well as its synthetic analogs—tecans) is a Topoisomerase I inhibitor. The activity of tecans against several breast cancer cell lines is well documented. One tecan drug, topotecan, was tested clinically in neoplastic carcinoma patients (Groves M D, Glantz M J, Chamberlain M C, Baumgartner K E, Conrad C A, Hsu S, Wefel J S, Gilbert M R, Ictech S, Hunter K U, Forman A D, Puduvalli V K, Colman H, Hess K R, Yung W K. Neuro-Oncology 2008, 10:208-15), but the outcome was not promising due to the poor pharmacokinetics of this (small molecule) drug in CSF. Tecans are not the only group of hydrophobic drugs that can be explored in the proposed neoplastic meningitis therapy approach, but the combination of significant background data on tecans and availability of the conjugate technology that fits the proposed approach makes camptothecin a suitable drug for the proposed study.

Based on the experimental data on CPT release from PHF-CPT, the action of the conjugate after intrathecal administration will be localized in the target compartment (leptomeningeal tissues contacting with CSF). The kinetics of CPT release includes two phases: (1) non-enzymatic release of a highly hydrophobic prodrug camptothecin succinimidoglycinate (CPT-SI), and (2) release of active CPT from CPT-SI (Scheme 6). CPT-SI is extremely hydrophobic and practically insoluble in water. Thus, it is expected that CPT-SI will be deposited in the leptomeningeal space and will redistribute only locally along cell membranes and other hydrophobic compartments. In tumors, CPT-SI released from the carrier was found to evenly distribute throughout the tissue. A similar distribution pattern in the meningeal compartment is expected, with CPT-SI penetration from CSF into the cancer cell formations through several cell layers.

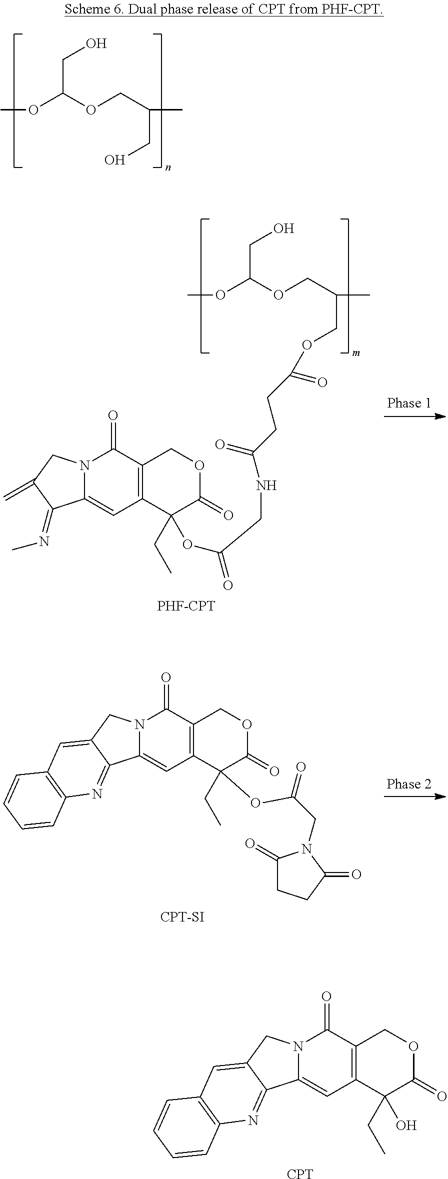

Penetration of CPT-SI from meninges to the systemic circulation is expected to be slow, and thus the conjugate is not expected to exert significant systemic toxicity. However, in view of some toxicity of tecans to non-cancer cells, some dose-dependent local toxicity with neurological and/or general manifestation can be expected.

Intrathecal MTD of PHF-CPT. The toxicity of the optimized PHF-CPT is evaluated in a dose elevation experiment. The conjugate is injected intrathecally in 20 g CD mice (n=3 per dose) in tripling dose increments. The animals are observed for 30 days for neurological signs (tremors, seizures, activity), weight dynamics and survival. The dose range in which 1 to 3 animals per group will die is covered by 50% dose increments (n=4 per dose) to estimate LD-50. The maximal dose at which all animals survive and the weight dynamics (if suppressed) returns to normal within 30 days is considered MTD.

The toxicity of the optimized PHF-CPT is evaluated in a dose elevation experiment. The conjugate is injected intrathecally in 100 g SD rats (n=3 per dose) in tripling dose increments. The systemic MTD of a preparation similar in chemical structure to PHF-CPT was found to be >24 mg/kg by CPT in mice (Rahier N J, Eisenhauer B M, Gao R, Thomas S J and Hecht S M. Bioorganic & Medicinal Chemistry 2005, 13:1381-86), but the initial distribution volume after the IT administration (cranial and spinal compartments) will be less than 10% of the distribution volume after the systemic administration. Therefore, the initial dose will be 1/10^{th} of the expected MTD corrected for the 10 fold smaller volume, i.e. 0.24 mg/kg by CPT. The animals are observed for 30 days for neurological signs (tremors, seizures, activity), weight dynamics and survival. The dose range in which 1 to 3 animals per group suffer severe toxicity are covered by 50% increments (n=4 per dose) to estimate the severely toxic dose. The maximal dose at which all animals survive, do not experience severe toxicity or ≧20% weight loss is considered the MTD.

CPT distribution in meninges after intrathecal administration of PHF-CPT. This experiment establishes the dynamics of CPT concentration in the target tissues, which is used to develop the optimal dose schedule in the final efficacy experiment. A fraction of the synthesized PHF-CPT conjugate is labeled with ¹²⁴I. For the purpose of this study, the iodination point in the camptothecin molecule is unimportant. Camptothecin is readily halogenated at carbon 7, and it is expected that a degree of iodination required for PET imaging under conditions preserving the conjugate structure (pH=7, t=25° C., iodination for 30 minutes with Iodogen or chloramin T) is readily achieved. If direct iodination does not result in a conjugate with desirable activity (ca. 5 mCi/mg), CPT is iodinated through replacement of the OH group at position 20 with iodine, and the resultant [¹²⁴I]iodo-CPT is incorporated into the conjugate during the synthesis. (The half-life of ¹²⁴I is 4 days, which allows for 60-80% activity retention during the Scheme 5 synthesis.)

The radiolabeled PHF-CPT conjugate is administered intrathecally at MTD into a group of rats (n=6). PET imaging is carried out as described above to evaluate CPT deposition and washout in the meningeal compartment over 8 days (dynamic imaging for 30 min after the injection, then static imaging at 2, 4, 8 hours and 2, 4 and 8 days).

Next, in a separate group of animals, the same dose of non-radioactive PHF-CPT is administered. The animals are euthanized at the time point at which 50% of the material is retained in the meningeal compartments as determined by PET. Rats are cryotomized across the brain, and the unstained, unfixed 15 μm sections are investigated by photoimaging to determine the microdistribution of camptothecin in meninges and brain parenchyma by the intrinsic fluorescence of camptothecin at 370 nm/440 nm.

PET imaging in monkeys (n=4) with CPT-labeled PHF-CPT can be carried out at a lower dose (mg/m² equivalent of 50% of mouse MTD, to ensure survival of the animals). Compared to the rat experiment, a more detailed data on CPT distribution in meninges is obtained due to the better resolution.

These studies (CPT retention, deposition and toxicity) provide the data that are useful to set the dose and injection schedule in the subsequent efficacy experiment and to project animal data to humans.

Efficacy of PHF-CPT. The efficacy of the optimized PHF-CPT conjugate is studied in a nude rat model (or nude mouse model with fluorescent subclone of SK-BR-3) of meningeal cancer spread utilizing human breast carcinoma SK-BR-3.

The study consists of two experiments. In the first one, the influence of a single dose on the dynamics of the disease is tested. In the second experiment, the data of the first experiment and the data from PET studies on CPT deposition are used to optimize the dose and injection schedule and to test the efficacy of PHF-CPT in a multiple injection protocol.

Single injection. The efficacy of single injection is studied in 3 groups of n=8 each, at three intrathecal doses: MTD, 0.5 MTD and 0.25 MTD. Control groups include untreated animals (n=8), animals treated with the same dose of PHF-CPT injected intravenously (n=8), and animals injected with an equimolar (by camptothecin) dose of free camptothecin injected intrathecally. Animals are injected PHF-CPT 4 days post implant and observed daily for toxicity signs and weight loss for the shorter of 60 days or 10% survival time.

Multiple injection. The multiple injection schedule is developed on the basis of the above single injection study and PET data on CPT deposition in the meningeal tissues to design a protocol that (1) establishes an effective concentration of CPT on the first injection, and (2) maintains the same concentration over a prolonged period of time. The data are plotted as survival dynamics vs. time and compared with controls.

The multiple injection schedule is tested in a group of n=12. An untreated group of the same size is used as a control. The first injection is made 4 days post implant at a dose that provided the most significant prolongation of survival in the single injection study. The follow-up doses are given at an interval corresponding to 50% reduction of camptothecin concentration in the meninges (as measured by PET). The follow-up dose is calculated, also using PET data, to return camptothecin concentration in the meninges to the initial level. The injections are continued at the same dose and at the same interval for the shorter of 60 days or 10% survival time. Animals are observed daily for signs of toxicity and weight loss. The data are plotted as survival dynamics vs. time and compared with controls.

Cell culture studies. Background data on the SK-BR-3 line sensitivity to PHF-CPT and CPT is carried out in 25% and 100% confluent cultures, in triplicate experiments, with concentration elevation from 1 nM in 2× increments. Cells are grown in McCoy's 5a medium supplemented with 10% FBS. Cells are seeded in 24-well culture plates (˜10000 cells/well), cultured until the desired level of confluence is achieved, then treated with model drugs. Growth inhibition is assessed 72 hours post treatment, using a commercial modified MTT photometric assay. The anti-proliferative effects are expressed as ID₅₀ values.

This strategy informs the practitioner on the function of leptomeningeal compartment in breast cancer, and allows for an evaluation of a model conjugate for leptomeningeal chemotherapy. The primate PET data enables one to scale the rodent data to humans. Thus, the study will lead to candidate chemotherapeutics suitable for scale-up and formal preclinical and human studies.

### Example 7

### Drug Delivery to Meninges and Peripheral Brain Parenchyma Utilizing Dual Phase Drug Release

This Example describes the development of drug forms suitable for targeting the subdural tissues (leptomeninges, brain, spinal cord, major nerves) through intrathecal administration. Despite the direct access to the target tissues, IT administration of conventional antineoplastic drugs was shown to be generally as inefficient as systemic administration, because of the fast washout in the highly vascularized arachnoid tissues (Fleischhack G, Jaehde U, and Bode U. Clin Pharmacokinet 2005; 44: 1-31). So far, IT administration was found to be effective only in two areas, where long drug presence in the target is not required: short-term anesthesia and diagnostic cisternography. The currently available chemotherapeutics are rapidly cleared from the CSF, and even the use of continuous drug administration or liposomal (Benesch M, Sovinz P, Krammer B, Lackner H, Mann G, Schwinger W, Gadner H, and Urban C. J Pediatr Hematol Oncol 2007; 29:222-6) formulations has not resulted in significant improvements.

The present disclosure encompasses the recognition that it would be useful for a hydrophilic agent that, after the IT administration through a lumbar port, would reach cranial as well as spinal CSF sub-compartments, and release a hydrophobic drug form that would not be washed out by the arachnoid vasculature.

As stated above, one of the problems hindering development of intrathecally administered drugs is the surprisingly incomplete knowledge on the drainage of CSF, clearance of solutes from it, and even on the general direction and rate of the CSF flow between the cranial and spinal regions. It has been proposed that CSF may drain either into the interstitium outside CNS at various locations and then into the lymphatic system (Walter B. A., Valera V. A., Takahashi S, and Ushiki T. Neuropathology and Applied Neurobiology (2006), 32, 388-396), while a fraction may be drained straight into the veins of the valve-like arachnoid granulations in the sagittal sinuses (Johnston M, Armstrong D and Koh L. Cerebrospinal Fluid Research 2007, 4:3-8). Briefly, Applicant's data suggest that: (1) there is no significant lymphatic drainage of CSF anywhere in the primate body; (2) the drainage of CSF to the blood is direct; (3) the CSF flow in the spinal column is predominantly downwards; (4) large molecule administered in the lumbar region effectively reach the cranial compartment being injected in a large (but clinically acceptable) volume; (5) being injected in a small volume, they may drain out of the CSF compartment faster than they propagate along the spinal cord anteriorly; and (6) protein clearance from CSF appears to be size-dependent at least in the lower molecular weight range (20-100 kDa), which suggest a second mechanism of protein clearance in addition to size-independent drainage with CSF in the arachnoid granulations.

It is envisioned that a macromolecular DPDR conjugate of an optimal hydrodynamic size can be efficiently delivered to all CSF compartments (with a possible exclusion of the ventricular sub-compartment) and release a hydrophobic drug that will distribute to the solid tissues and resist washout (Sarapa N, Britto M R, Speed W, Jannuzzo M G, Breda M, James C A, Porro M G, Rocchetti M, Wanders A, Mahteme H, Nygren P. Cancer Chemother Pharmacol (2003) 52: 424-430). The optimal intrathecal DPDR conjugate may likely have a larger hydrodynamic size than a previously developed CPT-PHF conjugate for intravenous administration (Yurkovetskiy A, Choi S, Hiller A, Yin M, McCusker C, Syed S, Fischman A J, and Papisov M. Biomacromolecules 2005, 6:2648-2658). In primates, the optimal release rate may likely be in single hours. In rodents, because of the much faster remixing of the CSF reservoir and faster CSF turnaround, the optimal release rate is expected to be under one hour. In this Example, the model conjugates are optimized for efficacy studies in rodents. Data scaling to the human PK should be made, preferably, on the basis of PK studies carried out in primates.

### Structures, Syntheses and Radiolabeling.

Macromolecular conjugates of CPT with PHF similar in the structure with previously tested structures (Sausville et al. Proceedings of AACR-NCI-EORTC International Conference Molecular Targets and Cancer Therapeutics, 2009 (November 2009) abstr B52) are synthesized and tested. Unlike previously studied conjugates, the conjugates are synthesized using a DPDR linker with the fastest known to date release rate, Gly-(2,2-dimethyl succinate) (half-release time of 36 min). Also, carrier molecules of four different hydrodynamic sizes (tentatively, 3, 5, 10 and 12 nm) are tested to determine whether large molecule clearance from CSF is (completely or in part) size-dependent. The conjugates with CPT content of ca. 5% w/w are synthesized, analyzed and radiolabeled as described in the previous Examples. All conjugates are subfractionated by gel chromatography to obtain preparations with narrow molecular size/weight distributions (tentatively, DPI<1.3).

### PET Imaging.

PET imaging is carried out as described above. The model conjugates are injected IT into cisterna magna in minimal volume (ca. 50 μl). The objective of the imaging is to determine, for each conjugate size, the patterns of the initial deposition of CPT, the fraction escaped to systemic circulation, and CPT washout kinetics in the meninges and in other deposition sites.

### Photoimaging.

Photoimaging of CPT fluorescence in unstained meninges and brain tissues is carried out as described above. The objective of imaging is to determine whether CPT deposition in meninges and pia mater is compartmentalized intra- or extracellularly, whether CPT translocates to brain parenchyma from the initial deposition sites, and to estimate the rate of translocation.

### Pharmacokinetics and Image Interpretation.

Based on Applicant's preliminary data obtained with proteins, the major fraction of the carrier will be contained in the CSF and thus the principal deposition site will be in the leptomeninges (generally, between the gray mater and dura mater, inclusively). We expect a minor fraction of the carrier to escape from CSF to the systemic circulation and release CPT in the blood. The amount of the systemically deposited CPT will be determined, and the systemic deposits will be compared with those formed after intravenous administration of the same preparation. Another minor fraction of CPT is expected to be deposited in brain parenchyma. The mechanism of large molecule transport from CSF to the parenchyma is unknown, but the evidence of such transport was detected in several studies, including Applicant's own. It was hypothesized that large molecules may propagate along longitudinal channels present uniquely in the walls of cerebral blood vessels (Rennels M, Gregory T F, Blaumanis O R, Fujimoto K, Grady P A. Brain Res. 1985, 326:47-53).

### Conjugate Selection.

A conjugate showing large initial deposition in the cerebral region (minimal clearance to the systemic circulation) is selected for detailed studies. If two or more conjugates show similar characteristics, other significant factors can be useful in selecting a candidate (e.g., the fraction deposited in the brain parenchyma and uniformity of distribution between the meningeal subcompartments, or the conjugate with the smallest molecular size may be advantageous for technological reasons such as lower viscosity and easier scaleup).

### Example 8

### Imaging with ¹²⁴I-Labeled PHF-CPT

In this Example, injection, imaging, and reconstruction protocols were used as described in the previous Examples. PHF-CPT conjugate was synthesized as described earlier, lyophilized and stored at −80° C. The lyophilized preparation was reconstituted at 30 mg/mL in 5 mM citrate buffered saline, pH=6. Then, 0.1 mL of the solution was mixed with 0.05 mL of 0.2 M sodium phosphate buffer solution, ph=7.5, and 10 mCi of carrier-free [I¹²⁴]NaI solution containing 0.5 mCi of ¹²⁴I in a Iodogen (Pierce) iodination tube. The reaction mixture was incubated for 5 minutes and the radiolabeled conjugate was desalted on a PD-10 column using 5 mM citrate buffered saline, pH=6, as an eluant. The macomolecular fractions were collected and distributed into three syringes containing 30±3 μCi of the conjugate (1 mg by weight) each.

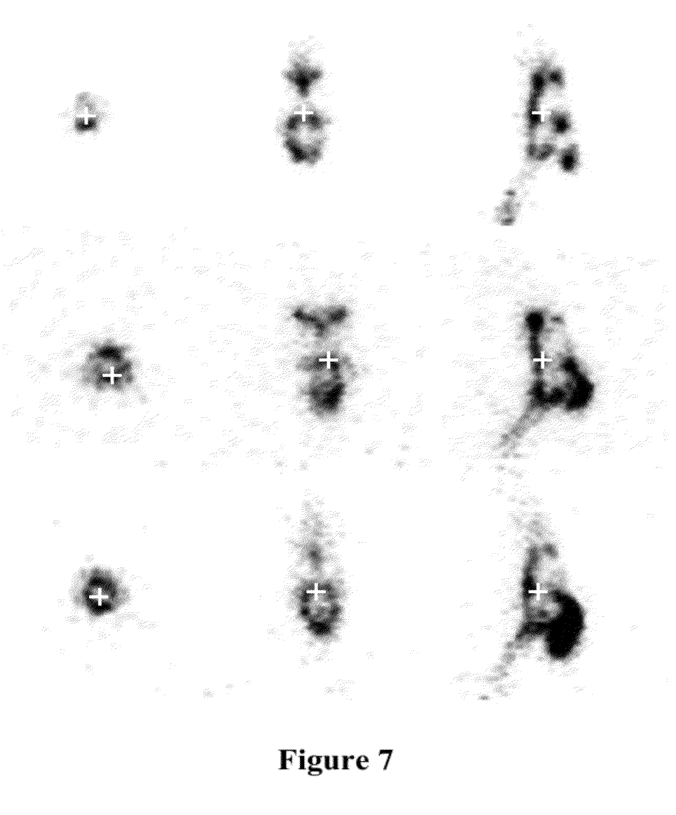

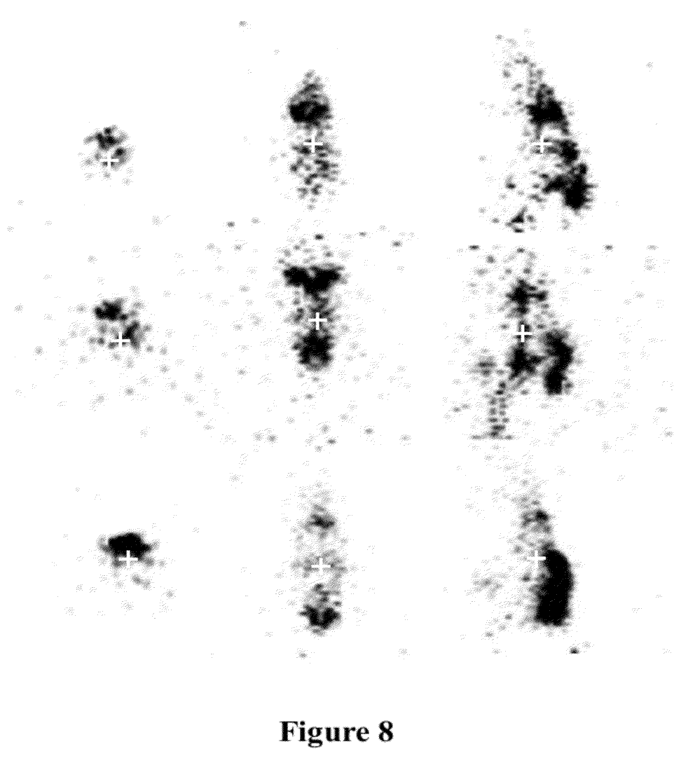

The conjugate was injected intrathecally (cysterna magna) into anesthetised rats weighing 550±50 g (n=3). The animals were imaged immediately after the injection and at 2 hours. FIG. 7 shows that the conjugate distributed into the entire cerebral CSF volume and partially into the spinal CSF immediately after the injection. FIG. 8 shows retention of the activity in the cerebral compartment 2 hours post injection. The duration of the imaging session was 20 minutes.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A method comprising the step of administering to an animal suffering from or susceptible to a cerebral, meningeal, or neural disease, disorder, or condition a conjugate comprising a carrier substituted with one or more occurrences of a moiety having the structure: 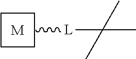
wherein:
each occurrence of M is independently a modifier; [symbol]
denotes direct or indirect attachment of M to linker L;
each occurrence of L is independently a linker; and
L is directly or indirectly attached to the carrier;
wherein the conjugate is administered directly into the cerebrospinal fluid space of the animal.

2. The method of claim 1, wherein the disease, disorder, or condition is a tumor of the brain or metastases of other primary tumors to the brain.

3. The method of claim 1, wherein the disease, disorder, or condition is selected from the group consisting of neoplastic meningitis, meningiomas, Alzheimer disease, geriatric conditions, neuropathies, lysosomal storage diseases, pain, and pernicious anemia.

4. A method comprising the step of administering to an animal suffering from or susceptible to an infection or infectious disease of the brain or CSF space a conjugate comprising a carrier substituted with one or more occurrences of a moiety having the structure: 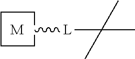
wherein:
each occurrence of M is independently a modifier; [symbol]
denotes direct or indirect attachment of M to linker L;
each occurrence of L is independently a linker; and
L is directly or indirectly attached to the carrier;
wherein the conjugate is administered directly into the cerebrospinal fluid space of the animal.

5. The method of claim 1, wherein the carrier is a polyacetal or polyketal.

6. The method of claim 5, wherein at least a subset of the polyacetal repeat structural units have the following chemical structure: 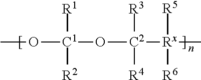
wherein for each occurrence of the n bracketed structure, one of R¹ and R² is hydrogen, and the other is a biocompatible group and contains a carbon atom covalently attached to C¹; R^{x} is a carbon atom covalently attached to C²; n is an integer; each occurrence of R³, R⁴, R⁵ and R⁶ is a biocompatible group and is independently hydrogen or an organic moiety; and for each occurrence of the bracketed structure n, at least one of R¹, R², R³, R⁴, R⁵ and R⁶ comprises a functional group suitable for coupling with a succinamide through an ester bond.

7. The method of claim 5, wherein at least a subset of the polyketal repeat structural units have the following chemical structure: 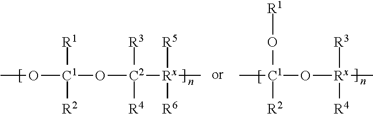
wherein each occurrence of R¹ and R² is a biocompatible group and contains a carbon atom covalently attached to C¹ or OC¹; R^{x} is a carbon atom covalently attached to C² or OC¹; n is an integer; each occurrence of R³, R⁴, R⁵ and R⁶ is a biocompatible group and is independently hydrogen or an organic moiety; and for each occurrence of the bracketed structure n, at least one of R¹, R², R³, R⁴, R⁵ and R⁶ comprises a functional group suitable for coupling with a succinamide through an ester bond.

8. The method of claim 6, wherein each occurrence of L is independently a moiety having the structure: 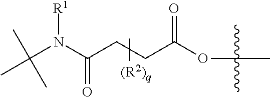
wherein: 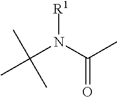
denotes the site of attachment to the modifier M; 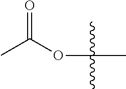
denotes the site of attachment to the carrier; p is an integer from 1-12; q is an integer from 0-4; R¹ is hydrogen, —C(═O)R^{1A}, —C(═O)OR^{1A}, —SR^{1A}, SO₂R^{1A} or an aliphatic, alicyclic, heteroaliphatic, heterocyclic, aryl, heteroaryl, aromatic, heteroaromatic moiety, wherein each occurrence of R^{1A} is independently hydrogen, alkyl, alkenyl, alkynyl, cycloalkyl, cycloalkenyl, cycloalkynyl, heteroalkyl, heteroalkenyl, heteroalkynyl, heterocycloalkyl, heterocycloalkenyl, heterocycloalkynyl, heteroaliphatic, heteroalicyclic, aromatic, heteroaromatic, aryl or heteroaryl; and each occurrence of R and R² is independently hydrogen, halogen, —CN, NO₂, an aliphatic, heteroaliphatic, aryl, heteroaryl, aromatic, heteroaromatic moiety, or -GR^{G1} wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, or —SO₂NR^{G2}—, wherein each occurrence of R^{G1}, R^{G2} and R^{G3} is independently hydrogen, halogen, or an optionally substituted aliphatic, heteroaliphatic, aromatic, heteroaromatic, aryl or heteroaryl moiety.

9. The method of claim 6, wherein each occurrence of L is independently a moiety having the structure: 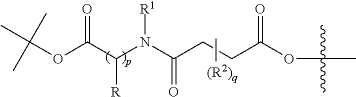
wherein 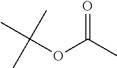
denotes the site of attachment to the modifier M; 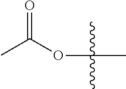
denotes the site of attachment to the carrier; p is an integer from 1-12; q is an integer from 0-4; R¹ is hydrogen, —C(═O)R^{1A}, —C(═O)OR^{1A}, —SR^{1A}, SO₂R^{1A} or an aliphatic, alicyclic, heteroaliphatic, heterocyclic, aryl, heteroaryl, aromatic, heteroaromatic moiety, wherein each occurrence of R^{1A} is independently hydrogen, alkyl, alkenyl, alkynyl, cycloalkyl, cycloalkenyl, cycloalkynyl, heteroalkyl, heteroalkenyl, heteroalkynyl, heterocycloalkyl, heterocycloalkenyl, heterocycloalkynyl, heteroaliphatic, heteroalicyclic, aromatic, heteroaromatic, aryl or heteroaryl; and each occurrence of R and R² is independently hydrogen, halogen, —CN, NO₂, an aliphatic, heteroaliphatic, aryl, heteroaryl, aromatic, heteroaromatic moiety, or -GR^{G1} wherein G is —O—, —S—, —NR^{G2}—, —C(—O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(NR^{G2})—, —NR^{G2}C(NR^{G3})—, —NR^{G2}SO₂—, —NR^{G2}SO₂NR^{G3}—, or —SO₂NR^{G2}—, wherein each occurrence of R^{G1}, R^{G2} and R^{G3} is independently hydrogen, halogen, or an optionally substituted aliphatic, heteroaliphatic, aromatic, heteroaromatic, aryl or heteroaryl moiety.

10. (canceled)

11. (canceled)

12. The method of claim 8, wherein each occurrence of L is independently a moiety having the structure: 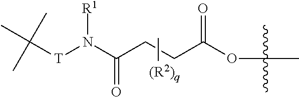
wherein: 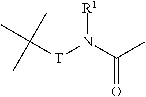
denotes the site of attachment to a modifier M;
T is a covalent bond or an optionally substituted, bivalent C₁₋₁₂ saturated or unsaturated, straight or branched, hydrocarbon chain, wherein one or more methylene units of L are independently replaced by -Cy-, —C(R^{x})₂—, —NR^{x}—, —N(R^{x})C(O)—, —C(O)N(R^{x})—, —N(R^{x})SO₂—, —SO₂N(R^{x})—, —O—, —C(O)—, —OC(O)—, —C(O)O—, —S—, —SO—, —SO₂—, —C(═S)—, —C(═NR^{x})—, —N═N—, or —C(═N₂)—;
each Cy is independently an optionally substituted bivalent ring selected from phenylene, a 3-7 membered saturated or partially unsaturated carbocyclylene, a 3-7 membered saturated or partially unsaturated monocyclic heterocyclylene having 1-2 heteroatoms independently selected from nitrogen, oxygen, or sulfur, or a 5-6 membered heteroarylene having 1-3 heteroatoms independently selected from nitrogen, oxygen; and
each R^{x} is independently hydrogen, a natural or unnatural amino acid side chain, or an optionally substituted group selected from C₁₋₆ aliphatic, phenyl, a 3-7 membered saturated or partially unsaturated carbocyclic ring, a 3-7 membered saturated or partially unsaturated monocyclic heterocyclic ring having 1-2 heteroatoms independently selected from nitrogen, oxygen, or sulfur, or a 5-6 membered heteroaryl ring having 1-3 heteroatoms independently selected from nitrogen, oxygen, or sulfur.

13-15. (canceled)

16. The method of claim 1, wherein one or more occurrences of M is an anchoring moiety.

17. The method of claim 16, wherein the one or more anchoring moieties is selected from the group consisting of idursulfase, arylsulfatase A, and sulfamidase.

18. The method of claim 1, wherein each occurrence of M is independently selected from the group consisting of biomolecules, small molecules, organic or inorganic molecules, therapeutic agents, detectable labels, microparticles, pharmaceutically useful groups or entities, macromolecules, DNA or RNA, anti-sense agents, gene vectors, virions, diagnostic labels, chelating agents, intercalator, hydrophilic moieties, dispersants, charge modifying agents, viscosity modifying agents, surfactants, coagulation agents and flocculants.

19. The method of claim 1, wherein one or more occurrences of M comprises a diagnostic label.

20. (canceled)

21. The method of claim 1, wherein one or more occurrences of M is a hydrophobic drug.

22. The method of claim 1, wherein one or more occurrences of M is a drug effective against cancer.

23-25. (canceled)

26. A method comprising the step of: administering to an animal suffering from or susceptible to a meningeal or neural disorder a conjugate comprising a carrier substituted with one or more occurrences of a moiety having the structure: 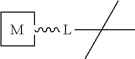
wherein:
each occurrence of M is independently a modifier; [symbol]
denotes direct or indirect attachment of M to linker L;
each occurrence of L is independently a linker; and
L is directly or indirectly attached to the carrier;
wherein the conjugate diffuses into the cerebrospinal fluid space of the animal via disease or injury-disrupted BBB.

27. A method for administering a conjugate comprising a carrier substituted with one or more occurrences of a moiety having the structure: 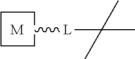
wherein:
each occurrence of M is independently a modifier; [symbol]
denotes direct or indirect attachment of M to linker L;
each occurrence of L is independently a linker; and
L is directly or indirectly attached to the carrier;
wherein the conjugate is administered directly into the cerebrospinal fluid space of an animal;
wherein the linker is characterized in that it releases the modifier into the cerebrospinal fluid space at a rate sufficient to provide an efficient amount of the modifier;
wherein the conjugate displays continued residence in the cerebrospinal fluid for at least 30 minutes.

28. A method of providing therapy or neuroprotection to an animal in need thereof for treating Alzheimer's disease, Parkinson's disease, amyotrophic lateral sclerosis, multiple sclerosis, HIV neuropathy, Guillain-Barre' syndrome, neural transplantation, neural xenotransplantation, stroke, brain hemorrhage, brain and spine trauma, ionizing radiation, neurotoxicity of vestibular structures, or retinal detachment, which comprises administering to said animal a conjugate comprising a carrier substituted with one or more occurrences of a moiety having the structure: 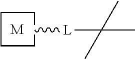
wherein:
each occurrence of M is independently a modifier; [symbol]
denotes direct or indirect attachment of M to linker L;
each occurrence of L is independently a linker; and
L is directly or indirectly attached to the carrier;
wherein the conjugate is administered directly into the cerebrospinal fluid space of an animal;
wherein the linker is characterized in that it releases the modifier into the cerebrospinal fluid space at a rate sufficient to provide an efficient amount of the modifier;
wherein the conjugate displays continued residence in the cerebrospinal fluid for at least 30 minutes.

29-35. (canceled)

36. A composition comprising a conjugate, wherein the conjugate comprises a carrier substituted with one or more occurrences of a moiety having the structure: 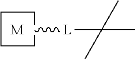
wherein:
each occurrence of M is independently a modifier; [symbol]
denotes direct or indirect attachment of M to linker L;
each occurrence of L is independently a linker; and
L is directly or indirectly attached to the carrier;
wherein one or more occurrences of M is independently a chemotherapeutic agent, a neuroprotective agent, an anti-infective agent, or a hydrophobic drug, with the proviso that M is not taxol or camptothecin.

37-39. (canceled)
